(19) United States
(12) Patent Application Publication
(10) Pub. No.: US 2013/0035307 A1
(43) Pub. Date: Feb. 7, 2013

(54) METHODS FOR TREATING OR PREVENTING THE SPREAD OF CANCER USING SEMI-SYNTHETIC GLYCOSAMINOGLYCOSAN ETHERS

(71) Applicants: Glenn D. Prestwich, Eastsound, WA (US); Thomas P. Kennedy, Charlotte, NC (US)

(72) Inventors: Glenn D. Prestwich, Eastsound, WA (US); Thomas P. Kennedy, Charlotte, NC (US)

(73) Assignee: UNIVERSITY OF UTAH RESEARCH FOUNDATION, Salt Lake City, UT (US)

(21) Appl. No.: 13/575,069

(22) PCT Filed: Jan. 24, 2011
(86) PCT No.: PCT/US2011/022218

§ 371 Date: Oct. 23, 2012

Related U.S. Application Data
(60) Provisional application No. 61/298,350, filed on Jan. 26, 2010.

(51) Int. Cl. A61K 31/728; A61P 35/00

(52) U.S. Cl. 514/54

(57) ABSTRACT

Described herein are methods for the treatment and prevention of tumor metastasis using alkylated and fluoroalkylated semi-synthetic glycosaminoglycan ethers (“SAGEs”). The synthesis of sulfated alkylated and fluoroalkylated SAGEs is also described.

### CROSS REFERENCE TO RELATED APPLICATIONS

This application claims priority upon U.S. provisional application Ser. No. 61/298,350, filed Jan. 26, 2010. This application is hereby incorporated by reference in its entirety.

### BACKGROUND

Trousseau's syndrome facilitates the hypercoagulable state of cancer and promotes efficient tumor metastasis. Trousseau's syndrome refers to the chronic disseminated coagulopathy and predisposition to deep venous thrombosis and pulmonary thromboembolism in patients with neoplasms (Sack G H Jr, et al. 1977). Trousseau's syndrome refers to any form of excessive coagulation in cancer (Varki A. 2007). Unlike venous thrombosis and thromboembolism from other causes, oral anticoagulants are often ineffective, and treatment with the anticoagulant heparin is often required to prevent thrombosis (Sack G H Jr, et al. 1977; Bell W R, et al. 1985; Levine M. 2002).

Tumor metastasis is inhibited in animal models by heparin and its derivatives. Heparin therapy is not only more successful than warfarin or other anticoagulants at preventing deep venous thromboembolism in patients with cancer, but is also useful in preventing metastasis. Although heparin and its derivatives have shown promise in preventing metastasis, treatment with heparin and its derivatives exhibits several major drawbacks. First, heparin and its derivatives are porcine-derived, thus leading to concerns of cross-species transfer of viruses. Second, because of heparin's anticoagulant properties, patients treated with this compound are at risk of excessive bleeding. Third, heparin may induce thrombocytopenia in certain individuals who produce an antibody to the complex of heparin with the cationic protein platelet factor-4 (PF-4), resulting in catastrophic platelet aggregation and generalized paradoxical arterial and venous clotting. Thus, an important unmet need is to formulate compounds that can be used to prevent metastasis while avoiding the myriad of side effects seen in other treatments.

### BRIEF SUMMARY

In accordance with the purpose of this invention, as embodied and broadly described herein, this invention relates to the treatment and prevention of tumor metastasis using alkylated and fluoroalkylated semi-synthetic glycosaminoglycan ethers (“SAGEs”). The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several embodiments of the disclosed methods and compositions and together with the description, serve to explain the principles of the disclosed methods and compositions.

FIG. 1 shows synthesis of selected methylated SAGEs.

FIG. 2A shows that methylated SAGEs inhibit P-selectin Inhibition of P-selectin glycoprotein ligand-1 (PSGL-1) binding to P-selectin by semi-synthetic glycosaminoglycan ethers (SAGEs) was studied using calcein-labeled U937 cells incubated in microwells coated with P-selectin. After 1 h, plates were washed, bound cells lysed and quantitated at λₑₓ=494 nm, λₑₘ=517 nm. The most potent is GM-112101, IC₅₀=17 ng/ml. This is consistent with the higher P-selectin inhibition with increased sulfation at C-6 of the N-acetylglucosamine residue.

FIG. 2B shows that SAGEs inhibit L-Selectin Inhibition of L-Selectin binding to receptors on U937 cells by semi-synthetic glycosaminoglycan ethers (SAGEs) was studied using calcein-labeled U937 cells incubated in microwells coated with L-Selectin. After 1 h, plates were washed, bound cells lysed and quantitated at λₑₓ=494 nm, λₑₘ=517 nm. The most potent to date is GM-111102, a compound equivalent in structure to GM-111101, shows an IC₅₀=57.7 ng/ml.

FIG. 3 shows that methylated SAGEs inhibit HLE activity. HLE (100 nM) was incubated with SAGEs at 1-100 nm concentrations in 0.5 M HEPES buffer for 15 min. Following incubation, the elastase substrate, Suc-Ala-Ala-Val-pNA was added to the reaction mixture to the final concentration of 0.3 mM. p-NA hydrolysis was followed for 15 min at absorbance of 405 nm. A very narrow range of IC₅₀ values in the range of 117-420 ng/ml was observed.

FIG. 4 shows that methylated SAGEs inhibit RAGE ligation. Microwell plates coated with CML-BSA (FIG. 9A), S100b calgranulin (FIG. 9B) or HMGB-1 (FIG. 9C) were incubated with RAGE-Fc chimera with or without SAGE for 2 h. Plates were washed, incubated with anti-RAGE Ab, incubated for 1 h, washed 4× and incubated with HRP-conjugated secondary Ab for 1 h. A colorimetric reaction was produced by addition of the chromogen TMB and quantitated by absorbance at 450 nm. FIG. 9A shows SAGEs inhibit AGE ligation of RAGE. FIG. 9B shows SAGEs inhibit S100 calgranulin ligation of RAGE. FIG. 9C shows SAGEs inhibit HMGB-1 ligation of RAGE.

FIG. 5 shows that methylated SAGEs show minimal or no activation of Factor XII. Pooled human plasma was incubated with heparin or a SAGE and amidolytic activity was determined using D-cyclohydrotyrosyl-Gly-Arg-p-NA

FIG. 6 shows that the SAGE GM-1111101 inhibits B16F10 melanoma lung metastasis model in C57/B 16 mice. Micrographs and numbers of melanin-laden (black) metastasis in the lungs of groups of six mice 28 days after B16F10 melanoma cells were administered i.v. 30 minutes after SAGEs, Heparin, and PBS subcutaneously treatment.

FIG. 7 shows the SAGE GM-111101 improves survival in B16F10 melanoma metastasis model.

FIG. 8 shows GM-111101 inhibition of A549 cancer cell migration using scratch wound assay.

FIG. 9 shows GM111101 inhibition of B16F10 melanoma cell migration using scratch wound assay.

FIG. 10 shows GM-111101 inhibition of HCT-116 metastatic colon cancer cell migration using scratch wound assay.

FIG. 11 shows GM111101 inhibition of MDA-MB-231 metastatic breast cancer cell migration using scratch wound assay.

FIG. 12 shows H&E staining for histology of lungs from treated and untreated mice in which metastases were generated by injection of B16F10 cells intravenously.

FIG. 13 shows an exemplary synthetic procedure for making alkylated and fluoroalkylated SAGEs.

### DETAILED DESCRIPTION

The disclosed methods and compositions may be understood more readily by reference to the following detailed description of particular embodiments and the Example included therein and to the Figures and their previous and following description.

Disclosed are materials, compositions, and components that can be used for, can be used in conjunction with, can be used in preparation for, or are products of the disclosed composition(s) and method(s). These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. Thus, if a class of molecules A, B, and C is disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F is specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed, it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the composition(s) and method(s) described herein. Such equivalents are intended to be encompassed by the appended claims.

It is understood that the disclosed composition(s) and method(s) are not limited to the particular methodology, protocols, and reagents described as these may vary. It is also to be understood that the terminology used herein is for the purpose of describing particular embodiments only and is not intended to limit the scope of the present invention which will be limited only by the appended claims.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Unless defined otherwise, all technical and scientific terms used herein have the same meanings as commonly understood by one of skill in the art to which the disclosed methods and compositions belong. Although any methods and materials similar or equivalent to those described herein can be used in the practice or testing of the present method and compositions, the particularly useful methods, devices, and materials are as described. Publications cited herein and the materials for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or can not be substituted and that the description includes both unsubstituted lower alkyl and lower alkyl where there is substitution.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” is also disclosed. It is also understood that the throughout the application, data are provided in a number of different formats, and that these data represent endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to” and is not intended to exclude, for example, other additives, components, integers or steps.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. For example, hyaluronan that contains at least one —OH group can be represented by the formula Y—OH, where Y is the remainder (i.e., residue) of the hyaluronan molecule.

The term “treat” as used herein is defined as maintaining or reducing the symptoms of a pre-existing condition. The term “prevent” as used herein is defined as eliminating or reducing the likelihood of the occurrence of one or more symptoms of a disease or disorder. The term “inhibit” as used herein is the ability of the compounds described herein to completely eliminate the activity or reduce the activity when compared to the same activity in the absence of the compound.

### A. COMPOSITIONS

1. SAGEs

Disclosed herein are alkylated and fluoroalkylated hyaluronan or derivatives thereof refered to herein as semi-synthetic glycosaminoglycan ethers (“SAGEs”) for use in the disclosed methods. The SAGE of the disclosed compositions and methods can comprise a modified hyaluronan or the pharmaceutically acceptable salt or ester thereof wherein at least one primary C-6 hydroxyl proton of the N-acetyl-glucosamine residue is substituted with an alkyl group or fluoroalkyl group. In some aspects, the SAGE of the disclosed compositions and methods can comprise a modified hyaluronan wherein at least one hydroxyl proton of the modified hyaluronan is substituted with a sulfate group.

In one aspect, at least one primary C-6 hydroxyl proton of the N-acetyl-glucosamine residue of hyaluronan is substituted with an alkyl group. The term “alkyl group” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. In one aspect, the alkyl group is a C₁-C₁₀ branched or straight chain alkyl group. In a further aspect, the alkyl group is methyl. The alkyl group can be unsubstituted or substituted. In the case when the alkyl group is substituted, one or more hydrogen atoms present on the alkyl group can be replaced with or more groups including, but not limited to, alkynyl, alkenyl, aryl, halide, nitro, amino, ester, ketone, aldehyde, hydroxy, carboxylic acid, aralkyl, or alkoxy.

In another aspect, at least one primary C-6 hydroxyl proton of the N-acetyl-glucosamine residue of hyaluronan is substituted with a fluoroalkyl group. The term “fluoroalkyl group” as used herein is a branched or unbranched saturated hydrocarbon group of 1 to 24 carbon atoms, wherein at least one of the hydrogen atoms is substituted with fluorine. In certain aspects, the fluoroalkyl group includes at least one trifluoromethyl group. In other aspects, the fluoroalkyl group has the formula —CH₂(CF₂)ₙCF₃, wherein n is an integer of 1, 2, 3, 4, 5, 6, 7, 8, 9, or 10. In one aspect, the fluoroalkyl group is —CH₂CF₂CF₃ or —CH₂CF₂CF₂CF₃.

In some aspects, the alkyl group of the disclosed SAGE comprises a C₁-C₁₀ branched or straight chain alkyl group. For example, the alkyl group can be a methyl, ethyl, propyl, iso-propyl, butyl, pentyl, or hexyl group. Thus, in some aspects, the alkyl group of the disclosed SAGE is a methyl group.

In some aspects, the fluoroalkyl group of the disclosed SAGE comprises at least one trifluoromethyl group. For example, the fluoroalkyl group can comprise the formula —CH₂(CF₂)ₙCF₃, wherein n is an integer from 0 to 10. Thus, in some aspects, n is 1, 2, 3, 4, or 5.

In some aspects, at least 1% of the primary C-6 hydroxyl protons of the N-acetyl-glucosamine residue are substituted with an alkyl group or fluoroalkyl group. For example, from 1% to 100% of the primary C-6 hydroxyl protons of the N-acetyl-glucosamine residue can be substituted with an alkyl group or fluoroalkyl group.

In some aspects, at least one C-2 hydroxyl proton or C-3 hydroxyl proton of the glucuronate residue or C-4 hydroxyl proton of the N-acetyl-glucosamine residue is substituted with an alkyl group or fluoroalkyl group.

In some aspects, the modified hyaluronan has a molecular weight greater than 10 kDa prior to alkylation or fluoroalkylation. For example, the modified hyaluronan can have a molecular weight from 40 kDa to 2,000 kDa prior to alkylation or fluoroalkylation.

In some aspects, at least one C-2 hydroxyl proton or C-3 hydroxyl proton of the glucuronate residue or C-4 hydroxyl proton of the N-acetyl-glucosamine residue is substituted with a sulfate group. In some aspects, at least one C-2 hydroxyl proton and C-3 hydroxyl proton of the glucuronate residue and the C-4 and/or C-6 hydroxyl protons of the N-acetyl-glucosamine residue is substituted with a sulfate group. For example, the C-2 hydroxyl proton and/or C-3 hydroxyl proton present on a glucuronic ring of hyaluronan can be substituted with a sulfate group. Alternatively, the C-4 hydroxyl and/or C-6 hydroxyl protons of the N-acetyl-glucosamine residue can be substituted with a sulfate group. In some aspects, the SAGE has a degree of sulfation from 0.5 to 3.5 per disaccharide unit.

In some aspects, the fluoroalkyl group of the disclosed SAGE is —CH₂CF₂CF₃ or —CH₂CF₂CF₂CF₃ and at least one C-2 hydroxyl proton and/or C-3 hydroxyl proton present on a glucuronic ring and/or C-4 hydroxyl proton or C-6 hydroxyl proton of the N-acetyl-glucosamine residue of hyaluronan is substituted with a sulfate group.

In some aspects, the hyaluronan or a derivative thereof from which the SAGE is produced is not derived from an animal source.

Table 1 provides the structures of several exemplary SAGEs. Each SAGE is identified by the code GM-XYSTZZ, where:

X=type of alkyl group, where 1=methyl, 2=pentafluoropropyl, 3=heptafluorobutyl, 4=benzylglycidyl ether

Y=size of HA, where 1=low, 2=medium, 3=high

S=degree of sulfation, where 1=partial, 2=full

T=degree of alkylation, where 1=low, 2=high

ZZ=sequential lot number 01 or 02, where the 02 has been made and has all the same properties as the 01 batch.

As used herein, “low” size HA refers to products obtained with HA starting materials between about 10 kDa to 100 kDa. As used herein, “medium” size HA refers to products obtained with HA starting materials between greater than 80 kDa to 300 kDa. As used herein, “high” size HA refers products obtained with HA starting materials between greater than 300 kDa to 2,000 kDa.

As used herein, low or partial sulfation levels refers to about 0.1 to about 1.5 sulfate groups per disaccharide. As used herein, full or high sulfation levels includes average sulfation levels greater than 1.5 sulfates per disaccharide.

As used herein, low alkylation levels refers to a degree of alkylation of about 0.1 to 1.0 per disaccharide. As used herein, high alkylation levels with degrees of alkylation greater than 1.0 per disaccharide.

TABLE 1
Structures of exemplary SAGEs

| | |
|---|---|
| GM-211101 GM-211201 GM-231101 GM-231201 GM-212101 GM-212201 GM-232101 GM-232201 | 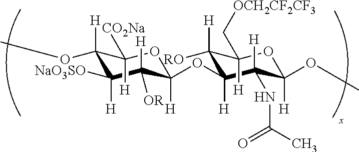 |
| GM-311101 GM-311201 GM-331101 GM-331201 GM-312101 GM-312201 GM-332101 GM-332201 | 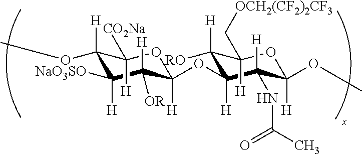 |
| GM-111101 GM-111201 GM-131101 GM-131201 GM-112101 GM-112201 GM-132101 GM-132201 | 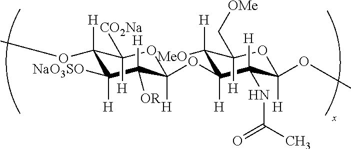 |

Table 2 provides a list of several SAGEs as defined by the code system above.

TABLE 2
Details of exemplary SAGEs

| | | MW | MW | | | |
|---|---|---|---|---|---|---|
| SAGE # | Chemical Name | (starting) | (GPC) | Alkylation | | Sulfation |
| GM-211101 | LMW-P-OSFHA-1(DS 1) | 53K | 6K | Pentafluoropropyl (Pfp) | 1 | 1.0-1.5 |
| GM-311101 | LMW-P-OSFHA-2(DS 1) | 53K | 5.8K | Heptafluorobutyl (Hfb) | 1 | 1.0-1.5 |
| GM-111101 | LMW-P-OSMeHA(DS 1) | 53K | 5.6k | Methyl (Me) | 1 | 1.0-1.5 |
| GM-211201 | LMW-P-OSFHA-1(DS 2) | 53K | 6K | pentafluoropropyl | 2 | 1.0-1.5 |
| GM-311201 | LMW-P-OSFHA-2(DS 2) | 53K | 5.6k | heptafluorobutyl | 2 | 1.0-1.5 |
| GM-111201 | LMW-P-OSMeHA(DS 2) | 53K | 5.5K | methyl | 2 | 1.0-1.5 |
| GM-231101 | P-OSFHA-1(DS 1) | 950K | 112k | Pentafluoropropyl | 1 | 1.0-1.5 |
| GM-331101 | P-OSFHA-2(DS 1) | 950K | 110k | Heptafluorobutyl (Hfb) | 1 | 1.0-1.5 |
| GM-131101 | P-OSMeHA(DS 1) | 950K | 123k | methyl | 1 | 1.0-1.5 |
| GM-231201 | P-OSFHA-1(DS 2) | 950K | 108k | pentafluoropropyl | 2 | 1.0-1.5 |
| GM-331201 | P-OSFHA-2(DS 2) | 950K | 130k | heptafluorobutyl | 2 | 1.0-1.5 |
| GM-131201 | P-OSMeHA(DS 2) | 950K | 120K | methyl | 2 | 1.0-1.5 |
| GM-212101 | LMW-F-OSFHA-1(DS 1) | 53K | 5k | pentafluoropropyl | 1 | 1.5-2.0 |
| GM-312101 | LMW-F-OSFHA-2(DS 1) | 53K | 4.8k | heptafluorobutyl | 1 | 1.5-2.0 |
| GM-112101 | LMW-F-OSMeHA(DS 1) | 53K | 5.6k | methyl | 1 | 1.5-2.0 |
| GM-212201 | LMW-F-OSFHA-1(DS 2) | 53K | 6K | pentafluoropropyl | 2 | 1.5-2.0 |
| GM-312201 | LMW-F-OSFHA-2(DS 2) | 53K | 6K | heptafluorobutyl | 2 | 1.5-2.0 |
| GM-112201 | LMW-F-OSMeHA(DS 2) | 53K | 5.4k | methyl | 2 | 1.5-2.0 |
| GM-232101 | F-OSFHA-1(DS 1) | 950K | 110k | pentafluoropropyl | 1 | 1.5-2.0 |
| GM-332101 | F-OSFHA-2(DS 1) | 950K | 105k | heptafluorobutyl | 1 | 1.5-2.0 |
| GM-132101 | F-OSMeHA(DS 1) | 950K | 112k | Methyl | 1 | 1.5-2.0 |
| GM-232201 | F-OSFHA-1(DS 2) | 950K | 120k | pentafluoropropyl | 2 | 1.5-2.0 |
| GM-332201 | F-OSFHA-2(DS 2) | 950K | 118k | heptafluorobutyl | 2 | 1.5-2.0 |
| GM-132201 | F-OSMeHA(DS 2) | 950K | 116K | methyl | 2 | 1.5-2.0 |
| GM-431101 | P-OSBGHA | 950K | 105k | benzyl glycidyl ether (BG) | <1 | ≈1 |
| GM-432101 | F-OSBGHA | 950K | 110k | benzyl glycidyl ether | <1 | ≈1 |
| GM-411101 | P-OSBGHA | 53K | 6K | benzyl glycidyl ether | <1 | ≈1 |
| GM-412101 | F-OSBGHA | 53K | 5.6k | benzyl glycidyl ether | <1 | ≈1 |

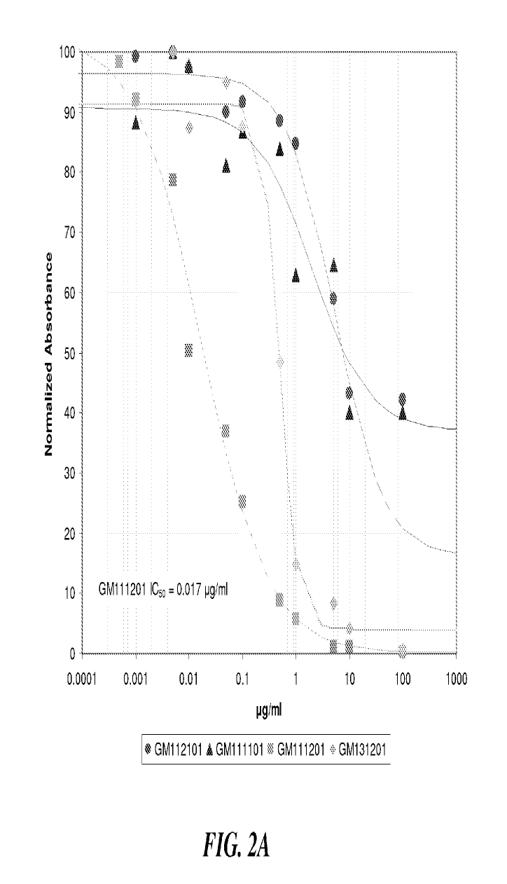

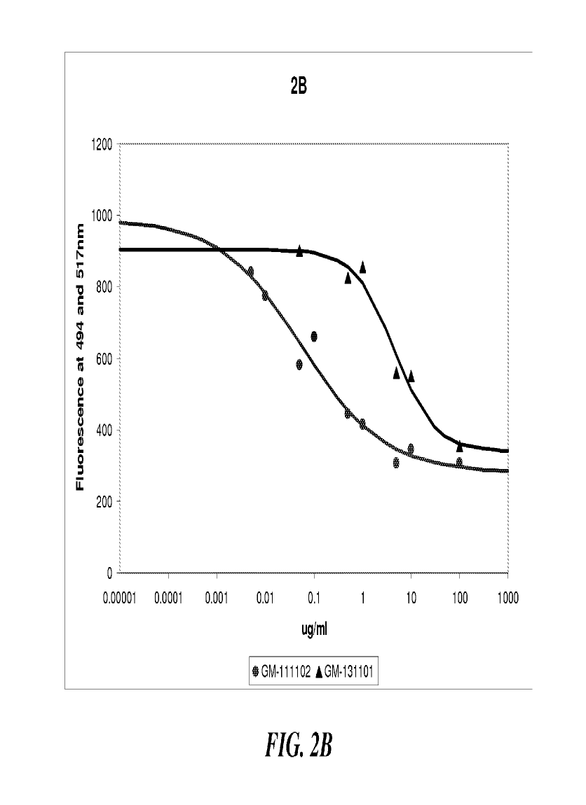

In one aspect, the alkyl group of the SAGE is methyl and at least one at least one C-2 hydroxyl proton, C-3 hydroxyl proton, C-4 hydroxyl proton, and/or C-6 hydroxyl proton of hyaluronan is substituted with a sulfate group. In another aspect, the alkyl group of the SAGE is methyl, at least one C-2 hydroxyl proton, C-3 hydroxyl proton, C-4 hydroxyl proton, and/or C-6 hydroxyl proton of hyaluronan is substituted with a sulfate group, and the compound has a molecular weight of 2 kDa to 200 kDa, such as 2 kDa to 10 kDa, after alkylation. An example of such a compound is GM-111101 as shown in FIG. 2.

Any of the alkylated and fluoroalkylated SAGEs described herein can be the pharmaceutically acceptable salt or ester thereof. In some aspects, the pharmaceutically acceptable ester or ester can be a prodrug. For example, free hydroxyl groups of SAGE GM-111101 can be partially esterified with palmitoyl chloride to afford an amphiphilic compound that is hydrolyzed by endogenous esterases to liberate the free SAGE. Other prosthetic groups that liberate non-toxic byproducts familiar to those skilled in the art may also be used. Pharmaceutically acceptable salts are prepared by treating the free acid with an appropriate amount of a pharmaceutically acceptable base. Representative pharmaceutically acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, benzalkonium, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C. such as at room temperature. The molar ratio of compounds of structural formula I to base used are chosen to provide the ratio desired for any particular salts. For preparing, for example, the ammonium salts of the free acid starting material, the starting material can be treated with approximately one equivalent of pharmaceutically acceptable base to yield a neutral salt.

Ester derivatives are typically prepared as precursors to the acid form of the compounds—as illustrated in the examples below—and accordingly can serve as prodrugs. Generally, these derivatives will be lower alkyl esters such as methyl, ethyl, and the like. Amide derivatives —(CO)NH₂, —(CO)NHR and —(CO)NR₂, where R is an alkyl group defined above, can be prepared by reaction of the carboxylic acid-containing compound with ammonia or a substituted amine. Also, the esters can be fatty acid esters. For example, the palmitic ester has been prepared and can be used as an alternative esterase-activated prodrug.

The SAGEs described herein can be formulated in any excipient the biological system or entity can tolerate to produce pharmaceutical compositions. Examples of such excipients include, but are not limited to, water, aqueous hyaluronic acid, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol. In certain aspects, the pH can be modified depending upon the mode of administration. For example, the pH of the composition is from about 5 to about 6, which is suitable for topical applications. Additionally, the pharmaceutical compositions can include carriers, thickeners, diluents, preservatives, surface active agents and the like in addition to the compounds described herein.

The pharmaceutical compositions can also include one or more active ingredients used in combination with the compounds described herein. The resulting pharmaceutical composition can provide a system for sustained, continuous delivery of drugs and other biologically-active agents to tissues adjacent to or distant from the application site. The biologically-active agent is capable of providing a local or systemic biological, physiological or therapeutic effect in the biological system to which it is applied. For example, the agent can act to control and/or prevent infection or inflammation, enhance cell growth and tissue regeneration, control tumor growth, act as an analgesic, promote anti-cell attachment, reduce alveolar bone and tooth loss, inhibit degeneration of cartilage and weight bearing joints, and enhance bone growth, among other functions. Additionally, any of the compounds described herein can contain combinations of two or more pharmaceutically-acceptable compounds. Examples of such compounds include, but are not limited to, antimicrobial agents, other antiinflammatory agents, other anticancer or antimetastatic agents, analgesics, anesthetics, and the like. Methods for using these compositions in drug delivery devices is described in detail below.

The pharmaceutical compositions can be prepared using techniques known in the art. In one aspect, the composition is prepared by admixing a SAGE described herein with a pharmaceutically-acceptable compound and/or carrier. The term “admixing” is defined as mixing the two components together so that there is no chemical reaction or physical interaction. The term “admixing” also includes the chemical reaction or physical interaction between the compound and the pharmaceutically-acceptable compound. Covalent bonding to reactive therapeutic drugs, e.g., those having nucleophilic groups, can be undertaken with the SAGEs, as in preparation of the prodrugs mentioned above. Second, non-covalent entrapment of a pharmacologically active agent in a cross-linked or non-crosslinked polysaccharide matrix is also possible. Third, electrostatic and/or hydrophobic interactions can facilitate retention of a pharmaceutically-acceptable compound in the compounds described herein.

It will be appreciated that the actual preferred amounts of SAGE in a specified case will vary according to the specific compound being utilized, the particular compositions formulated, the mode of application, and the particular situs and subject being treated. Dosages for a given host can be determined using conventional considerations, e.g. by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physicians Desk Reference, Barnhart Publishing (1999).

2. Combination Therapies

The SAGE disclosed herein can be used alone or in combination with one or more known or newly discovered bioactive. For example, the provided composition(s) can further comprise one or more of classes of antibiotics (e.g., Aminoglycosides, Cephalosporins, Chloramphenicol, Clindamycin, Erythromycins, Fluoroquinolones, Macrolides, Azolides, Metronidazole, Penicillins, Tetracyclines, Trimethoprim-sulfamethoxazole, Vancomycin), steroids (e.g., Andranes (e.g., Testosterone), Cholestanes (e.g., Cholesterol), Cholic acids (e.g., Cholic acid), Corticosteroids (e.g., Dexamethasone), Estraenes (e.g., Estradiol), Pregnanes (e.g., Progesterone), narcotic and non-narcotic analgesics (e.g., Morphine, Codeine, Heroin, Hydromorphone, Levorphanol, Meperidine, Methadone, Oxydone, Propoxyphene, Fentanyl, Methadone, Naloxone, Buprenorphine, Butorphanol, Nalbuphine, Pentazocine), anti-inflammatory agents (e.g., Alclofenac, Alclometasone Dipropionate, Algestone Acetonide, alpha Amylase, Amcinafal, Amcinafide, Amfenac Sodium, Amiprilose Hydrochloride, Anakinra, Anirolac, Anitrazafen, Apazone, Balsalazide Disodium, Bendazac, Benoxaprofen, Benzydamine Hydrochloride, Bromelains, Broperamole, Budesonide, Carprofen, Cicloprofen, Cintazone, Cliprofen, Clobetasol Propionate, Clobetasone Butyrate, Clopirac, Cloticasone Propionate, Cormethasone Acetate, Cortodoxone, Decanoate, Deflazacort, Delatestryl, Depo-Testosterone, Desonide, Desoximetasone, Dexamethasone Dipropionate, Diclofenac Potassium, Diclofenac Sodium, Diflorasone Diacetate, Diflumidone Sodium, Diflunisal, Difluprednate, Diftalone, Dimethyl Sulfoxide, Drocinonide, Endrysone, Enlimomab, Enolicam Sodium, Epirizole, Etodolac, Etofenamate, Felbinac, Fenamole, Fenbufen, Fenclofenac, Fenclorac, Fendosal, Fenpipalone, Fentiazac, Flazalone, Fluazacort, Flufenamic Acid, Flumizole, Flunisolide Acetate, Flunixin, Flunixin Meglumine, Fluocortin Butyl, Fluorometholone Acetate, Fluquazone, Flurbiprofen, Fluretofen, Fluticasone Propionate, Furaprofen, Furobufen, Halcinonide, Halobetasol Propionate, Halopredone Acetate, Ibufenac, Ibuprofen, Ibuprofen Aluminum, Ibuprofen Piconol, Ilonidap, Indomethacin, Indomethacin Sodium, Indoprofen, Indoxole, Intrazole, Isoflupredone Acetate, Isoxepac, Isoxicam, Ketoprofen, Lofemizole Hydrochloride, Lomoxicam, Loteprednol Etabonate, Meclofenamate Sodium, Meclofenamic Acid, Meclorisone Dibutyrate, Mefenamic Acid, Mesalamine, Meseclazone, Mesterolone, Methandrostenolone, Methenolone, Methenolone Acetate, Methylprednisolone Suleptanate, Momiflumate, Nabumetone, Nandrolone, Naproxen, Naproxen Sodium, Naproxol, Nimazone, Olsalazine Sodium, Orgotein, Orpanoxin, Oxandrolane, Oxaprozin, Oxyphenbutazone, Oxymetholone, Paranyline Hydrochloride, Pentosan Polysulfate Sodium, Phenbutazone Sodium Glycerate, Pirfenidone, Piroxicam, Piroxicam Cinnamate, Piroxicam Olamine, Pirprofen, Prednazate, Prifelone, Prodolic Acid, Proquazone, Proxazole, Proxazole Citrate, Rimexolone, Romazarit, Salcolex, Salnacedin, Salsalate, Sanguinarium Chloride, Seclazone, Sermetacin, Stanozolol, Sudoxicam, Sulindac, Suprofen, Talmetacin, Talniflumate, Talosalate, Tebufelone, Tenidap, Tenidap Sodium, Tenoxicam, Tesicam, Tesimide, Testosterone, Testosterone Blends, Tetrydamine, Tiopinac, Tixocortol Pivalate, Tolmetin, Tolmetin Sodium, Triclonide, Triflumidate, Zidometacin, Zomepirac Sodium), or anti-histaminic agents (e.g., Ethanolamines (like diphenhydrmine carbinoxamine), Ethylenediamine (like tripelennamine pyrilamine), Alkylamine (like chlorpheniramine, dexchlorpheniramine, brompheniramine, triprolidine), other anti-histamines like astemizole, loratadine, fexofenadine, Bropheniramine, Clemastine, Acetaminophen, Pseudoephedrine, Triprolidine).

Numerous anti-cancer (antineoplastic) drugs are available for combination with the present methods and compositions. Antineoplastic drugs include Acivicin, Aclarubicin, Acodazole Hydrochloride, AcrQnine, Adozelesin, Aldesleukin, Altretamine, Ambomycin, Ametantrone Acetate, Aminoglutethimide, Amsacrine, Anastrozole, Anthramycin, Asparaginase, Asperlin, Azacitidine, Azetepa, Azotomycin, Batimastat, Benzodepa, Bicalutamide, Bisantrene Hydrochloride, Bisnafide Dimesylate, Bizelesin, Bleomycin Sulfate, Brequinar Sodium, Bropirimine, Busulfan, Cactinomycin, Calusterone, Caracemide, Carbetimer, Carboplatin, Carmustine, Carubicin Hydrochloride, Carzelesin, Cedefingol, Chlorambucil, Cirolemycin, Cisplatin, Cladribine, Crisnatol Mesylate, Cyclophosphamide, Cytarabine, Dacarbazine, Dactinomycin, Daunorubicin Hydrochloride, Decitabine, Dexormaplatin, Dezaguanine, Dezaguanine Mesylate, Diaziquone, Docetaxel, Doxorubicin, Doxorubicin Hydrochloride, Droloxifene, Droloxifene Citrate, Dromostanolone Propionate, Duazomycin, Edatrexate, Eflomithine Hydrochloride, Elsamitrucin, Enloplatin, Enpromate, Epipropidine, Epirubicin Hydrochloride, Erbulozole, Esorubicin Hydrochloride, Estramustine, Estramustine Phosphate Sodium, Etanidazole, Ethiodized Oil I 131, Etoposide, Etoposide Phosphate, Etoprine, Fadrozole Hydrochloride, Fazarabine, Fenretinide, Floxuridine, Fludarabine Phosphate, Fluorouracil, Flurocitabine, Fosquidone, Fostriecin Sodium, Gemcitabine, Gemcitabine Hydrochloride, Gold Au 198, Hydroxyurea, Idarubicin Hydrochloride, Ifosfamide, Ilmofosine, Interferon Alfa-2a, Interferon Alfa-2b, Interferon Alfa-n1, Interferon Alfa-n3, Interferon Beta-I a, Interferon Gamma-Ib, Iproplatin, Irinotecan Hydrochloride, Lanreotide Acetate, Letrozole, Leuprolide Acetate, Liarozole Hydrochloride, Lometrexol Sodium, Lomustine, Losoxantrone Hydrochloride, Masoprocol, Maytansine, Mechlorethamine Hydrochloride, Megestrol Acetate, Melengestrol Acetate, Melphalan, Menogaril, Mercaptopurine, Methotrexate, Methotrexate Sodium, Metoprine, Meturedepa, Mitindomide, Mitocarcin, Mitocromin, Mitogillin, Mitomalcin, Mitomycin, Mitosper, Mitotane, Mitoxantrone Hydrochloride, Mycophenolic Acid, Nocodazole, Nogalamycin, Ormaplatin, Oxisuran, Paclitaxel, Pegaspargase, Peliomycin, Pentamustine, Peplomycin Sulfate, Perfosfamide, Pipobroman, Piposulfan, Piroxantrone Hydrochloride, Plicamycin, Plomestane, Porfimer Sodium, Porfiromycin, Prednimustine, Procarbazine Hydrochloride, Puromycin, Puromycin Hydrochloride, Pyrazofurin, Riboprine, Rogletimide, Safmgol, Safingol Hydrochloride, Semustine, Simtrazene, Sparfosate Sodium, Sparsomycin, Spirogermanium Hydrochloride, Spiromustine, Spiroplatin, Streptonigrin, Streptozocin, Strontium Chloride Sr 89, Sulofenur, Talisomycin, Taxane, Taxoid, Tecogalan Sodium, Tegafur, Teloxantrone Hydrochloride, Temoporfin, Teniposide, Teroxirone, Testolactone, Thiamiprine, Thioguanine, Thiotepa, Tiazofurin, Tirapazamine, Topotecan Hydrochloride, Toremifene Citrate, Trestolone Acetate, Triciribine Phosphate, Trimetrexate, Trimetrexate Glucuronate, Triptorelin, Tubulozole Hydrochloride, Uracil Mustard, Uredepa, Vapreotide, Verteporfin, Vinblastine Sulfate, Vincristine Sulfate, Vindesine, Vindesine Sulfate, Vinepidine Sulfate, Vinglycinate Sulfate, Vinleurosine Sulfate, Vinorelbine Tartrate, Vinrosidine Sulfate, Vinzolidine Sulfate, Vorozole, Zeniplatin, Zinostatin, Zorubicin Hydrochloride.

Other anti-neoplastic compounds include: 20-epi-1,25 dihydroxyvitamin D3, 5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, atrsacrine, anagrelide, anastrozole, andrographolide, angiogenesis inhibitors, antagonist D, antagonist G, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, canarypox IL-2, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chlorines, chloroquinoxaline sulfonamide, cicaprost, cis-porphyrin, cladribine, clomifene analogues, clotrimazole, collismycin A;, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexifosfamide, dexrazoxane, dexverapamil, diaziquone, didemnin B, didox, diethylnorspermine, dihydro-5-azacytidine, dihydrotaxol, 9-dioxamycin, diphenyl spiromustine, docosanol, dolasetron, doxifluridine, droloxifene, dronabinol, duocannycin SA, ebselen, ecomustine, edelfosine, edrecolomab, eflornithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, fmasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-like growth factor-1 receptor inhibitor, interferon agonists, interferons, interleukins, iobenguane, iododoxorubicin, ipomeanol, 4-irinotecan, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogues, mitonafide, mitotoxin fibroblast growth factor-saporin, mitoxantrone, mofarotene, molgramostim, monoclonal antibody, human chorionic gonadotrophin, monophosphoryl lipid A+myobacterium cell wall sk, mopidamol, multiple drug resistance genie inhibitor, multiple tumor suppressor 1-based therapy, mustard anticancer agent, mycaperoxide B, mycobacterial cell wall extract, myriaporone, N-acetyldinaline, N-substituted benzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, O6-benzylguanine, octreotide, okicenone, oligonucleotides, onapristone, ondansetron, ondansetron, oracin, oral cytokine inducer, ormaplatin, osaterone, oxaliplatin, oxaunomycin, paclitaxel analogues, paclitaxel derivatives, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylene conjugate, raf antagonists, raltitrexed, ramosetron, ras farnesyl protein transferase inhibitors, ras inhibitors, ras-GAP inhibitor, retelliptine demethylated, rhenium Re 186 etidronate, rhizoxin, ribozymes, RII retinamide, rogletimide, rohitukine, romurtide, roquinimex, rubiginone B1, ruboxyl, safingol, saintopin, SarCNU, sarcophytol A, sargramostim, Sdi 1 mimetics, semustine, senescence derived inhibitor 1, sense oligonucleotides, signal transduction inhibitors, signal transduction modulators, single chain antigen binding protein, sizofiran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitor, stem-cell division inhibitors, stipiamide, stromelysin inhibitors, sulfmosine, superactive vasoactive intestinal peptide antagonist, suradista, suramin, swainsonine, synthetic glycosaminoglycans, tallimustine, tamoxifen methiodide, tauromustine, tazarotene, tecogalan sodium, tegafur, tellurapyrylium, telomerase inhibitors, temoporfin, temozolomide, teniposide, tetrachlorodecaoxide, tetrazomine, thaliblastine, thalidomide, thiocoraline, thrombopoietin, thrombopoietin mimetic, thymalfasin, thymopoietin receptor agonist, thymotrinan, thyroid stimulating hormone, tin ethyl etiopurpurin, tirapazamine, titanocene dichloride, topotecan, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozole, zanoterone, zeniplatin, zilascorb, zinostatin stimalamer.

The compositions provided herein can further comprise one or more additional radiosensitizers. Examples of known radiosensitizers include gemcitabine, 5-fluorouracil, pentoxifylline, and vinorelbine. (Zhang et al., 1998; Lawrence et al., 2001; Robinson and Shewach, 2001; Strunz et al., 2002; Collis et al., 2003; Zhang et al., 2004).

3. Preparation of SAGEs

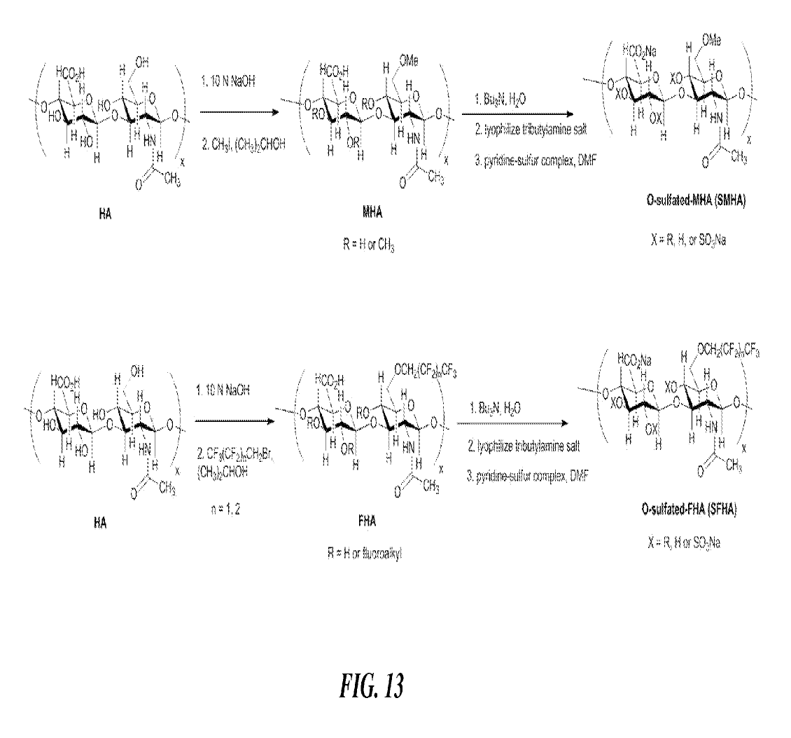

Described herein are methods for alkylating or fluoroalkylating hyaluronan to produce the precursors to the SAGEs. In one aspect, the SAGEs are produced by (a) reacting the hyaluronan or a derivative thereof with a sufficient amount of base to deprotonate at least one primary C-6 hydroxyl proton of the N-acetyl-glucosamine residue, and (b) reacting the deprotonated hyaluronan or a derivative thereof with an alkylating agent or fluoroalkylating for a sufficient time and concentration to alkylate or fluoroalkylate at least one deprotonated primary C-6 hydroxyl group. It will be understood by those skilled in the art that the basic conditions may also lead to cleavage of the glycosidic linkage, leading to lower molecular weight hyaluronan derivatives during the modification process. It will also be understood that the basic conditions deprotonate the acid to the carboxylate, and the secondary hydroxyl groups, and that each of these nucleophilic moieties may participate in the ensuing alkylation in proportion to their relative abundance at equilibrium and the nucleophilicity of the anionic species. For example, 2-O and/or 3-O hydroxyl and/or 4-OH hydroxyl protons can be deprotonated and alkylated or fluoroalkylated. An example of this is depicted in FIG. 13, where R can be hydrogen, an alkyl group, or an alkyl group.

The hyaluronan starting material can exist as the free acid or the salt thereof. Derivatives of hyaluronan starting material can also be used herein. The derivatives include any modification of the hyaluronan prior to the alkylation or fluoroalkylation step. A wide variety of molecular weight hyaluronan can be used herein. In one aspect, the hyaluronan has a molecular weight greater than 10 kDa prior to alkylation or fluoroalkylation. In another aspect, the hyaluronan has a molecular weight from 25 kDa to 1,000 kDa, 100 kDa to 1,000 kDa, 1000 kDa to 8000 kDa, 25 kDa to 500 kDa, 25 kDa to 250 kDa, or 25 kDa to 100 kDa prior to alkylation or fluoroalkylation. In certain aspects, the hyaluronan starting material or a derivative thereof is not derived from an animal source. In these aspects, the hyaluronan can be derived from other sources such as bacterial fermentation. For example, a recombinant B. subtilis expression system can be used to produce the hyaluronan starting material. In another aspect, a streptococcus strain can be used to produce the hyaluronan starting material.

The hyaluronan starting material or derivative thereof is initially reacted with a sufficient amount of base to deprotonate at least one primary C-6 hydroxyl proton of the N-acetyl-glucosamine residue. The selection of the base can vary. For example, an alkali hydroxide such as sodium hydroxide or potassium hydroxide can be used herein. The concentration or amount of base can vary depending upon the desired degree of alkylation or fluoroalkylation. In one aspect, the amount of base is sufficient to deprotonate and result in subsequent alkylation of at least 0.001% of the primary C-6 hydroxyl protons of the N-acetyl-glucosamine residues of the hyaluronan starting material or derivative thereof. In other aspects, the amount of base is sufficient to deprotonate and result in subsequent alkylation of from 0.001% to 50%, 1% to 50%, 5% to 45%, 5% to 40%, 5% to 30%, 5% to 20%, 10% to 50%, 20% to 50%, or 30% to 50% of the primary C-6 hydroxyl protons of the N-acetyl-glucosamine residue of the hyaluronan starting material or derivative thereof. It is understood that the more basic the solution, the more likely are chain cleavage reactions and the higher the degree of alkylation/fluoroalkylation that can be achieved. For example, other hydroxyl groups present on hyaluronan (e.g., 2-OH and/or 3-OH and/or 4-OH can be alkylated or fluoroalkylated). In one aspect, all of the hydroxyl groups present on hyaluronan can be alkylated or fluoroalkylated. In other aspects, 0.001%, 0.01%, 0.1%, 1%, 5%, 10%, 20%, 30%, 40%, 50%, 60%, 70%, 80%, 90%, 95%, 100%, or any range thereof of hydroxyl protons present on hyaluronan can be deprotonated and subsequently alkylated or fluoroalkylated. The degree of alkylation can vary depending upon base concentration, HA concentration, time, temperature and alkylating agent concentration. Exemplary conditions and procedures for alkylating HA are provided in the Examples.

After the hyaluronan starting material or derivative thereof has been treated with a base, the deprotonated hyaluronan is reacted with an alkylating agent or fluoroalkylating agent to produce the SAGE. Examples of alkylating agents include, but are not limited to, an alkyl halide. Alkyl bromides and iodides are particularly useful. Other leaving groups such as tosylates, mesylates, and triflates may also be useful. Similarly, the fluoroalkylating agent can include a fluoroalkyl halide. Alkylating agents and fluoroalkylating agents commonly used in organic synthesis can be used herein. One skilled in the art will also recognize that the basic alkylation conditions also include the possibility for beta-elimination of the halide and a proton from an adjacent carbon, if available. For this reason, the selection of alkylating agents that will not undergo beta-elimination, e.g., methyl halides, trifluoroethyl halides, and benzyl halides, are particularly useful herein.

An exemplary synthetic procedure for making alkylated and fluoroalkylated SAGEs is provided in FIG. 13. Referring to FIG. 13, hyaluronan (HA) is treated with a base (e.g., NaOH) and an alkylating agent (e.g., CH₃I) to methylate a primary C-6 hydroxyl proton of the N-acetyl-glucosamine residue of hyaluronan and produce methylated hyaluronan (MHA). FIG. 13 also provides an exemplary synthetic procedure for making a fluoroalkylated hyaluronan (FHA) using a fluoroalkylating agent (e.g., CF₃(CF₂)ₙCH₂Br).

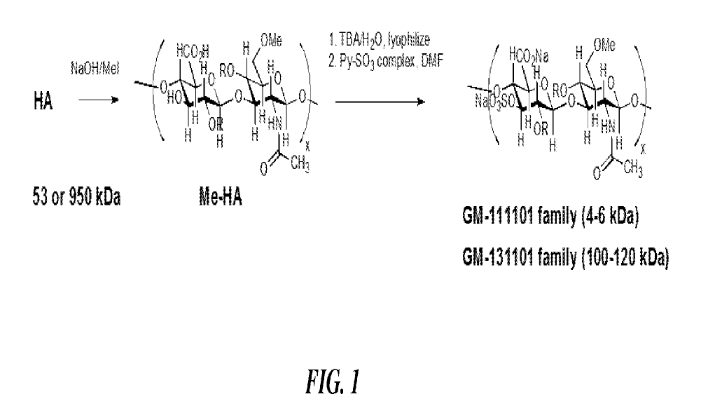

In certain aspects, it is desirable to sulfate the alkylated or fluoroalkylated SAGEs described above. In one aspect, the alkylated or fluoroalkylated SAGE is sulfated by reacting the alkylated or fluoroalkylated SAGE with a sulfating agent to produce a sulfated product. The degree of sulfation can vary from partial sulfation to complete sulfation. In general, free hydroxyl groups present on the alkylated or fluoroalkylated hyaluronan or a derivative thereof can be sulfated. In one aspect, at least one C-2 hydroxyl proton and/or C-3 hydroxyl proton of the glucuronate residue or the C-4 or C-6 hydroxyl protons of the N-acetyl-glucosamine residue is substituted with a sulfate group. Not wishing to be bound by theory, the sulfation of a partially C-6 alkylated SAGE at the C-6 hydroxyl group ensures that the SAGE retains P-selectin and L-selectin inhibitory potency. In another aspect, the degree of sulfation is from 0.1, 0.5, 1.0, 1.5, 2.0, 2.5, 3.0, 3.5 or any range thereof per disaccharide unit of the alkylated or fluoroalkylated SAGE. In one aspect, the alkylated or fluoroalkylated SAGE can be treated with a base to deprotonate one or more hydroxyl protons followed by the addition of the sulfating agent. The sulfating agent is any compound that reacts with a hydroxyl group or deprotonated hydroxyl group to produce a sulfate group. The molecular weight of the SAGE can vary depending upon reaction conditions. In one aspect, the molecular weight of the SAGE is from 1 kDa to 8000 kDa, 1 kDa to 2000 kDa, 2 kDa to 500 kDa, 2 kDa to 250 kDa, 2 kDa to 100 kDa, 2 kDa to 50 kDa, 2 kDa to 25 kDa, or from 2 kDa to 10 kDa. FIG. 1 depicts an exemplary synthesis of sulfated alkylated or fluoroalkylated SAGEs (SMHA and SFHA, respectively).

### B. METHODS

1. Preventing Metastasis

Provided herein is a method of treating cancer in a subject, comprising administering to the subject a composition comprising a SAGE disclosed herein. By “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Without wishing to be bound by theory, the disclosed compositions can treat cancer by inter alia preventing the spread of the cancer, i.e., tumor metastasis. Thus, also disclosed is a method of preventing tumor metastasis in a subject, comprising administering to the subject a composition comprising a SAGE disclosed herein. As used herein, “preventing” refers to a reduction or delay in the onset of metastasis and does not require absolute preclusion. Thus, also disclosed herein is a method of reducing the onset or severity of tumor metastasis in a subject, comprising administering to the subject a composition comprising a SAGE disclosed herein.

In some aspects, the disclosed methods comprise administering to a patient having a tumor, or undergoing surgery to resect a tumor, a composition comprising a SAGE disclosed herein.

In preferred aspects, the SAGEs used in the disclosed methods inhibit a selectin (e.g., P-selectin, L-selectin), heparanase, and/or RAGE activities but have low anti-coagulant activities compared to heparin. Also disclosed is a method of selecting SAGE molecules for use in the disclosed therapeutic methods based on screening for the ability to inhibit a selectin (e.g., P-selectin, L-selectin), heparanase, and/or RAGE activities but have low anti-coagulant activity compared to heparin. The skilled artisan can also routinely select preferred SAGE molecules for use the in the therapeutic methods based on other properties, such as safety and stability.

In some aspects, the SAGE of the disclosed therapeutic method has the partial structure as depicted in the tetrasaccharide fragment shown below:

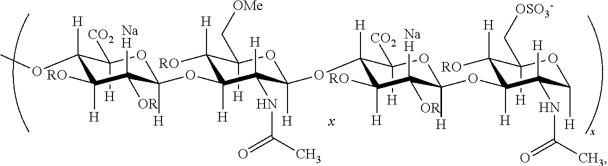

wherein R is H or SO₃Na. In some aspects, the starting HA is about 50 kDa or about 950 kDa. In some aspects, the methylation is from about 10% to about 200%. In some aspects, the sulfation level is low (about 0.5 to 1.0 per disaccharide). In some aspects, the sulfation level is high (greater than 2 per disaccharide). As depicted above, it is possible that the SAGE is not only alkylated at the C-6 hydroxyl group, but also the C-6 hydroxyl group can also be sulfated as well depending upon the conditions for producing the alkylated SAGE. In another aspect, all of the 6-OH hydroxyl groups are completely alkylated.

In some aspects, the SAGEs useful in the methods described herein are produced from HA having a molecular weight of about 50-65 kDa. In some aspects, the SAGE is produced from HA having a molecular weight of about 150-250 kDa, or about 950 kDa, or about 1,300 kDa. Starting molecular weights of HA from bacterial or animal sources may be lower than 50 kDa or higher than 2000 kDa, which can also be converted to SAGEs with the desired therapeutic properties. It will be appreciated by one skilled in the art that lower molecular weight SAGEs will be more permeable to the skin when applied topically, but would likely be cleared more rapidly when administered intravenously or subcutaneously. A higher molecular weight SAGE would not be topically effective, but when administered parenterally would be cleared more slowly. This results in a longer period of effectiveness in the body, where the SAGE would likely be cleaved by the body into biologically active lower molecular weight SAGEs during normal metabolism. To inhibit selectins effectively and block metastatic spread of cancers, SAGEs will be constructed of optimum molecular size. In one aspect, the SAGEs have a molecular weight greater than or equal 5 kDa.

The cancer of the disclosed methods can be any cell in a subject undergoing or subject to metastasis. A representative but non-limiting list of cancers that the disclosed compositions can be used to treat include bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, and pancreatic cancer. In addition, papopustular eruptions arising as side effects of chemotherapy with growth factor inhibitors, in particular EGFR inhibitors such as cetuximb, elotinib, and gefitinib, may be accessible to treatment with topical SAGEs in patients undergoing chemotherapy.

The SAGEs can prevent the spread of cancer cells from tumors. For example, the SAGE can be administered subcutaneously or intravenously prior to a surgical tumor resection, during the resection, and after the resection to limit attachment of any tumor cells released during surgery. In other aspects, the SAGE can be be administered subcutaneously or intravenously either prophyllactically or therapeutically to patients not undergoing oncological surgery to limit the spread of the disease.

2. Administration

The disclosed compounds and compositions can be administered in any suitable manner. The manner of administration can be chosen based on, for example, whether local or systemic treatment is desired, and on the area to be treated. For example, the compositions can be administered orally, parenterally (e.g., intravenous, subcutaneous, intraperitoneal, or intramuscular injection), by inhalation, extracorporeally, topically (including transdermally, ophthalmically, vaginally, rectally, intranasally) or the like.

As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

In another aspect, the SAGEs can be delivered topically via a transdermal patch such as, for example, a microneedle array. In this aspect, this mode of administration would be useful for the chronic treatment of cancer patients.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The exact amount of the compositions required can vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. Thus, effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage can vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products.

For example, a typical daily dosage of the SAGE disclosed herein used alone might range from about 1 μg/kg to up to 200 mg/kg of body weight or more per day, depending on the factors mentioned above and the mode of administration. In one aspect, the dosage is in the range of 1 mg/kg to 50 mg/kg, or 3 mg/kg to 30 mg/kg.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### SAGEs can be Conveniently and Inexpensively Produced from HA

A series of semi-synthetic glycosaminoglycan ethers (SAGEs, synthesized as shown in FIG. 1) were designed with several important concepts in mind. First, an immunoneutral starting polysaccharide, hyaluronic acid (HA), was employed. The HA disaccharide consists of long polymers of the disaccharide N-acetylglucosamine (GlcNAc) and glucuronic acid (GlcA) linked GlcNacβ1-3GlcAβ1-4 in repeating units along the chain, having the structure:

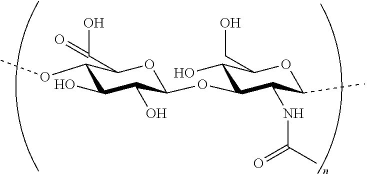

HA is abundant in skin, skeletal tissues, umbilical cord, synovial fluid, and especially the vitreous of the eye. A typical polymer may consist of 10,000 disaccharides and have masses up to 10,000 kDa. As a viscoelastic solution, HA confers rigidity to tissues when high concentrations of high molecular weight HA are present, but is elastic and has the physical property of a biologic lubricant, reducing friction when present in the joint space. HA is commercially available from a recombinant B. subtilis expression system (Novozymes Biopolymers) or from numerous suppliers using streptococcal fermentation strains (e.g., LifeCore). This bacterial sourcing of HA, whether from B. subtilis or from one of several commonly used strains of Streptococcus, improves the safety of SAGEs over polysaccharides such as heparin.

Sulfate analogues were produced to vary the amount of negative charge on the polymer. Sulfation can be adjusted from low (less than or equal to 0.5 per disaccharide) to high (up to 3.5 per disaccharide) to adjust the level of polyanionic charge and the anti-inflammatory properties it confers. Increased sulfation is known to increase anti-coagulant activities, and for this reason the level of sulfation in SAGEs must be carefully controlled to maintain the safety and anti-coagulant profile. Further, selecting HA also allowed for examination of a range of molecular sizes from less than or about 50 kDa to greater than or about 1,300 kDa. Finally, since sulfated polysaccharides are hydrophilic, the ether modification adds partial lipophilicity to the SAGEs and additionally reduces hydrolysis by hyaluronidases.

Each of these goals was met by developing a novel combination of alkylation and sulfation that creates a class of compounds referred to herein as SAGEs. Methylation of the primary hydroxyl occurs preferentially. The remaining hydroxyls can be hydroxyls or sulfates, depending on the level of alkylation and sulfation. Thus, the chemistry and size of the SAGEs can be adjusted to vary in vitro efficacy and in vivo depth of penetration into the skin. The synthesis and pharmacologic assessment of over 28 different SAGEs has been completed, including at least two specific families of methylated SAGEs based on molecular weight. For example, four are derived from 950 kDa HA and four from 53 kDa HA. One skilled in the art will recognize that different lots of HA may have slightly different average molecular weights and different size distributions, or polydispersity. Despite the differences in starting materials, SAGEs prepared from, for example, 67 kDa HA will have properties very similar to those prepared from 53 kDa HA. Similarly, SAGEs prepared from 1,300 kDa HA will have properties similar to those prepared from 950 kDa HA. The eight methylated SAGEs that investigated in the following example are summarized in Table 3. The potency, safety, and efficacy of these compounds are described below. As controls, the in vitro effects of heparin were also examined.

TABLE 3
Chemical Structures of Methylated SAGEs Synthesized

| | | Starting | Final | | | |
|---|---|---|---|---|---|---|
| SAGE | Old Chemical | Molecular | Molecular | | Alkylation | Sulfation |
| Number | Name | Weight | Weight | Alkylation | SD | SD |
| GM-111101 | LMW-P-OSMeHA | 53 kDa | 5.6 kDa | methyl | 1 | 1.0-1.5 |
| GM-111201 | LMW-P-OSMeHA | 53 kDa | 5.5 kDa | methyl | 2 | 1.0-1.5 |
| GM-131101 | P-OSMeHA | 950 kDa | 123 kDa | methyl | 1 | 1.0-1.5 |
| GM-131201 | P-OSMeHA | 950 kDa | 120 kDa | methyl | 2 | 1.0-1.5 |
| GM-112101 | LMW-F-OSMeHA | 53 kDa | 5.6 kDa | methyl | 1 | 1.5-2.0 |
| GM-112201 | LMW-F-OSMeHA | 53 kDa | 5.4 kDa | methyl | 2 | 1.5-2.0 |
| GM-132101 | F-OSMeHA | 950 kDa | 112 kDa | methyl | 1 | 1.5-2.0 |
| GM-132201 | F-OSMeHA | 950 kDa | 116 kDa | methyl | 2 | 1.5-2.0 |
| Heparin | heparin | 16 kDa | 16 kDa | n/a | 0 | 4-5 |

(LMW = low molecular weight; P = partial; F = fully; S = sulfated; Me = methyl; SD = substitution degree)

### 2. Example 2

### SAGEs Inhibit P-Selectin, Block Proteolytic Activity of Cationic PMN Proteases, and Disrupt the Interaction of RAGE with its Ligands

HA has intrinsic effects on inflammatory responses. Whereas HA fragments (Gao F, et al. 2008) can actually trigger inflammation by interaction with the cell surface Toll-Like Receptors (TLR) 2 and 4 (Jiang D, et al. 2007), intra-articular injection of high molecular weight HA is used for the pain and inflammation of osteoarthritis (Juni P, et al. 2007). In contrast, SAGEs are intrinsically anti-inflammatory, showing activities similar to heparin.

First, SAGEs are potent inhibitors of P-selectin and L-selectin. The same selectins discussed as important in tumor thrombogenesis and metastasis are also the initial adhesion molecules used by PMNs, monocytes and lymphocytes to marginate and roll along the blood vessel wall until binding such targets as the intercellular adhesion molecule-1 (ICAM-1). The competitor-mediated displacement of U937 human monocytes, which firmly adhere to P-selectin through P-selectin glycoprotein ligand-1 (PSGL-1), was studied using fluorescent-labeled cells. FIG. 2A shows that SAGEs inhibit U937 binding to P-selectin with 50% inhibitory concentrations (IC₅₀) in the ng/ml range. FIG. 2B shows analogous results for L-selectin. Because platelet adhesion to tumor cells is critically dependent upon the activity of P-selectin, this activity indicates that SAGEs can inhibit tumor metastasis by blocking the ability of tumor cells to migrate through the circulation free from immune surveillance (Stevenson J L, et al. 2007). Surprisingly, the most highly methylated SAGE, GM-111201 was the most potent; additional sulfation appears to reduce P-selectin binding (GM-112101).

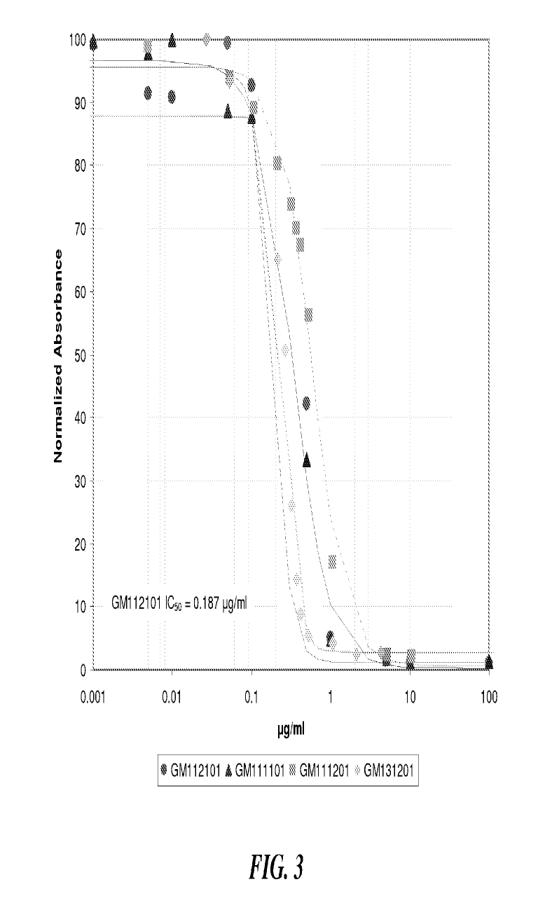

Second, as highly sulfated polyanions, SAGEs are potent inhibitors of PMN proteases such as human leukocyte elastase. FIG. 3 shows that SAGEs inhibit the PMN protease human leukocyte elastase (HLE) with IC₅₀ values in the nanomolar range. This indicates that SAGEs, acting as polyanions, can charge-neutralize cationic molecules such as neutrophil proteases via electrostatic interactions. A very narrow range of IC₅₀ values in the range of 117-420 ng/ml was observed for SAGE inhibition of HLE.

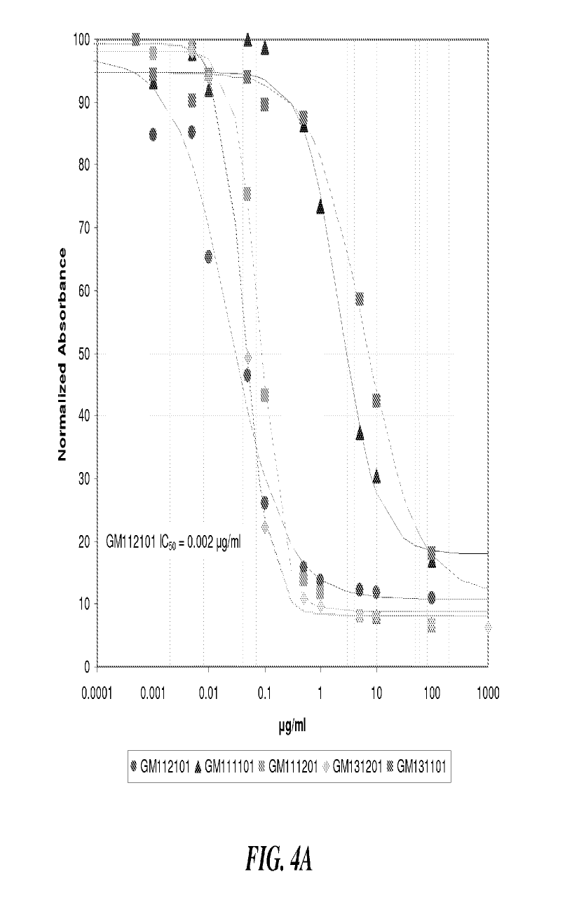

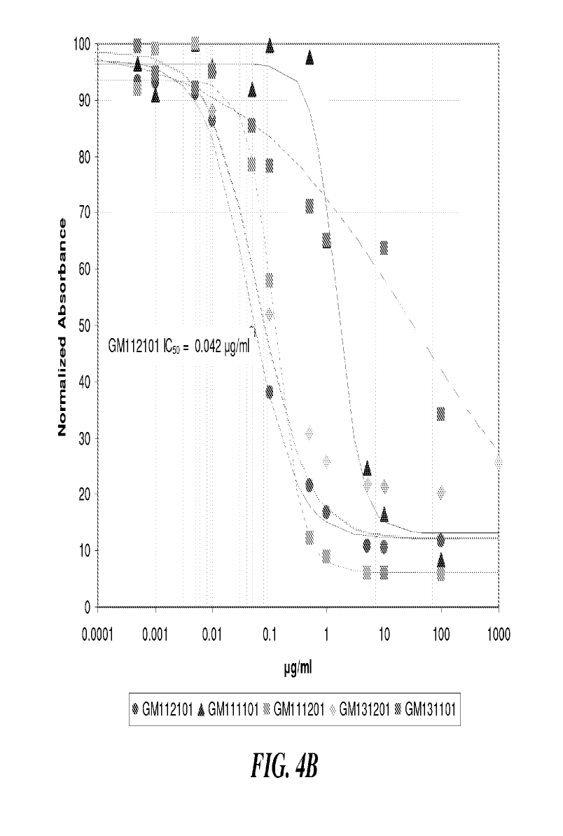

Third, SAGEs are extremely potent inhibitors of the Receptor for Advanced Glycation End-products (RAGE) with all of its ligands. The AGE product carboxymethyl lysine (CML)-modified protein is prominently formed in diabetes (Schmidt A M, et al. 2001; Bierhaus A, et al. 2005; Ramasamy R, et al. 2005) but also plays a role in melanoma proliferation, migration and invasion (Abe R, et al. 2004). The high MW SAGE GM-131101 potently inhibited interaction of CML-BSA with RAGE (FIG. 4A). The SAGE GM-112101 (a potent P-selectin inhibitor) was most potent in this assay, with IC₅₀=2 ng/ml; the higher mass GM131101 and GM 131201 were similar in potency.

Fourth, S100 calgranulins are small, calcium-binding, cell signaling molecules (Schmidt A M, et al. 2001; Bierhaus A, et al. 2005; Ramasamy R, et al. 2005) that have been shown to promote cancer proliferation and metastasis in an autocrine fashion (Logsdon C D, et al. 2007). The SAGEs inhibit ligation of RAGE by S100b calgranulin (FIG. 4B), which is highly expressed in melanoma (Harpio R, et al. 2004) Inhibition of these ligand-RAGE interactions occur over a broad range from ng/ml to μg/ml concentrations, with GM-112101 showing the highest potency with IC₅₀=42 ng/ml.

Finally, SAGEs inhibit ligation of RAGE by HMGB-1 (FIG. 4C), a nuclear protein that is released into the extracellular environment by cancer cells to facilitate cell motility and metastasis (Ellerman J E, et al. 2007). SAGEs inhibit the ability of monocytes and lymphocytes to ligate RAGE on vascular endothelium with the Mac-1 (CD11a/18b) counter-ligand (Chavakis T, et al. 2003) and use RAGE as an adhesion molecule essential for exiting the circulation into areas of inflammation. The SAGEs are slightly less potent than heparin, with GM-111101 and GM-112101 again showing the highest potencies (IC₅₀=455 and 537 ng/ml, respectively).

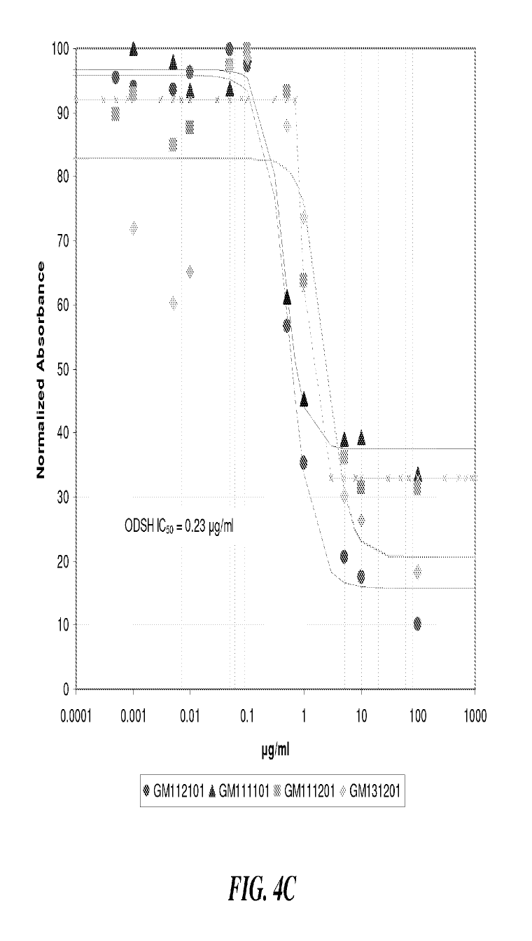

Heparin and its derivatives also effectively inhibit P-selectin, HLE and ligation of RAGE by its multiple ligands (14,16,59,68), but heparin is relatively expensive and can be adulterated during manufacture (Kakkar A K, et al. 2004; Lee A Y Y, et al. 2005). In contrast, SAGEs are not only less costly to produce but safer. SAGEs are also non-anticoagulant; those tested to date show no anti-Xa and <0.2 U/mg anti-Ba anticoagulant activities, compared to 150 U/mg each for unfractionated heparin. Unlike heparin, highly-charged polyanionic polymers are potent inducers of the intrinsic coagulation cascade by activation of Factor XII, secondarily activating kinins (Kishimoto T K, et al. 2008; Guerrini M, et al. 2008). SAGEs were thus screened for their ability to stimulate intrinsic coagulation (activation of Hagemann factor). Methylated SAGEs GM-111101 and GM-131101 appear safer than medical heparin in tests for activation of Factor XII, even at concentrations 10-100-fold higher than needed to achieve pharmacologic inhibition of selectins and RAGE (FIG. 5).

The in vitro test data for the methylated SAGEs, compared with heparin, are shown in Table 4. Bold values indicate the most potent derivative for a given assay. Selection of a therapeutic agent for any given therapeutic use will require balancing safety with efficacy in vivo.

TABLE 4
Activities of Methylated SAGEs (50% Inhibitory Concentrations (IC₅₀) in μg/ml)

| SAGE | P-Selectin/ | RAGE/ | RAGE/ | RAGE/ | RAGE/ | | Factor |
|---|---|---|---|---|---|---|---|
| Number | PSGL | Mac-1 | CML-BSA | S100B | HMGB1 | HLE | XII |
| GM-111101 | 0.14 | 0.042 | 2.27 | 1.56 | 0.455 | 0.22 | NR |
| GM-111201 | 0.017 | 0.033 | 0.082 | 0.12 | 1.033 | 0.58 | 0.4 |
| GM-131101 | 5.61 | 0.51 | 6.09 | 31.069 | TBD | 0.285 | NR |
| GM-131201 | 0.5 | 0.3 | 0.044 | 0.06 | 1.66 | 0.42 | 0.4 |
| GM-112101 | 2.164 | 0.113 | 0.002 | 0.042 | 0.537 | 0.187 | 0.4 |
| GM-112201 | 0.496 | TBD | 0.005 | 0.017 | 0.634 | 0.117 | 0.4 |
| GM-132101 | TBD | TBD | 0.015 | 0.004 | 0.501 | 0.127 | 0.4 |
| GM-132201 | 0.22 | 0.004 | 0.1 | 0.04 | TBD | 0.24 | 0.4 |
| Heparin | 0.3 | 0.11 | 0.39 | 1.29 | 0.04 | 0.21 | 0.4 |

NR = No Reaction;
TBD = to be determined

### 3. Example 3

### SAGEs are Safe Parenteral Agents with Low Toxicity

In preliminary experiments, it was shown that SAGEs have no toxicity for cultured normal fibroblasts and epithelial cells (keratinocytes) and exhibit no cutaneous toxicity in standard Draize tests. A study was completed on the effects of the GM-111101 administered as a single i.v. dose to rats, and also to evaluate the toxicity of GM-111101 with daily i.v. injections for a period of seven days at a single dose level (n=3 animals per sex per SAGE). GM-111101 did not produce signs of toxicity at any of the dose levels evaluated, including single acute doses of 3, 10, 30, and 100 mg/kg and seven repeated i.v. daily doses of 10 mg/kg. Therefore, the no observable effect level (NOEL) for i.v. GM-111101 in rats is at least 100 mg/kg. Due to the absence of mortality observed at all doses of GM-111101, the intravenous LD50 in rats for GM-111101 is considered to be greater than 100 mg/kg. These results indicate that SAGE GM-111101 will be safe to employ as systemic or injected treatments for diseases.

### 4. Example 4

### SAGEs Inhibit Tumor Implantation and Lung Metastasis in a Mouse Model

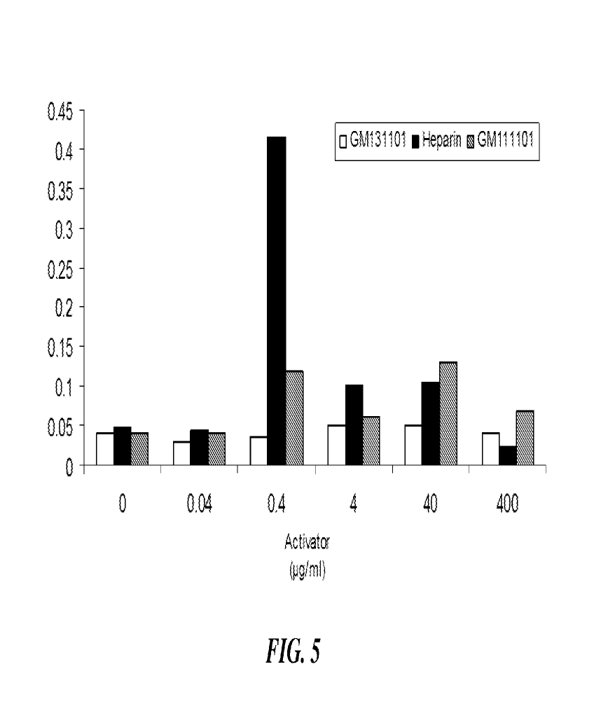

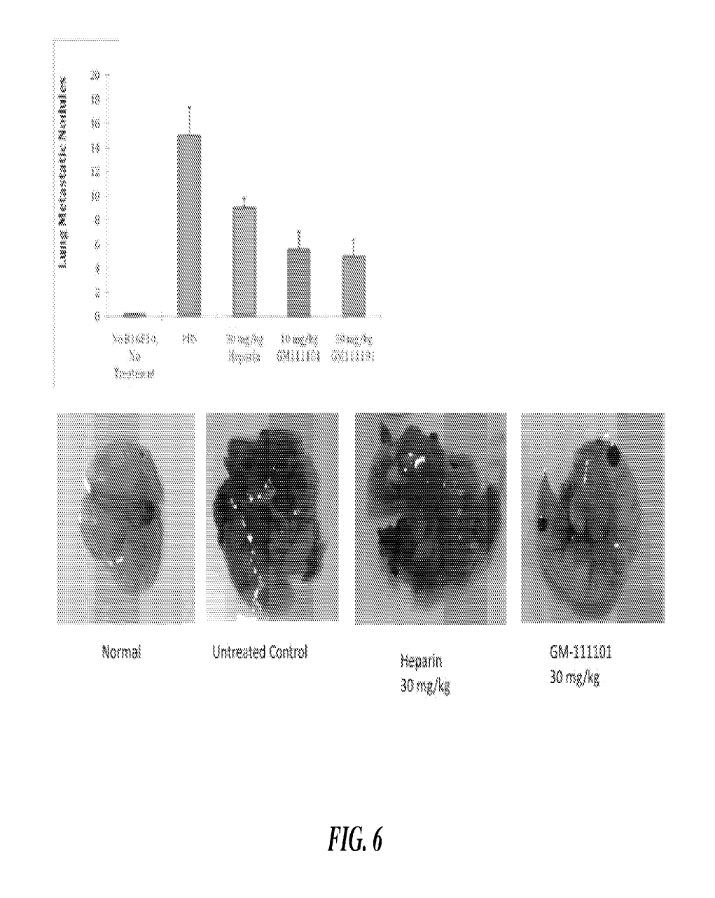

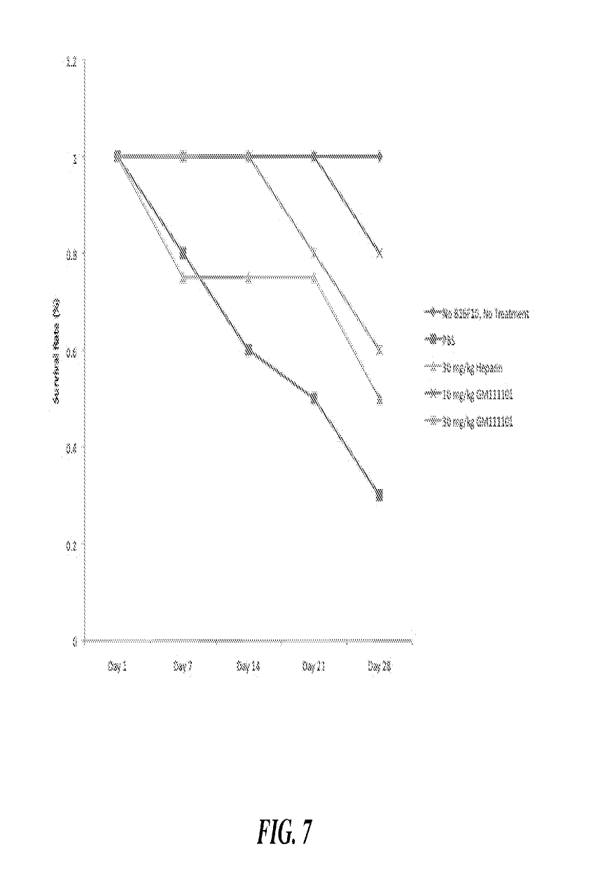

Using a standard model that tests the ability of heparins to inhibit tumor implantation and lung metastasis (Stevenson J L, et al. 2005), studies were performed on the anti-metastatic activity of SAGEs in vivo. C57B1/6 mice were injected subcutaneously with 100 μL of PBS, heparin (30 mg/kg), the SAGE GM-111101 (10 or 30 mg/kg). Thirty minutes afterwards, 500,000 B16F1 melanoma cells were injected i.v. into the lateral tail vein. Twenty-seven days after injection, the mice were euthanized, the lungs were removed, and numbers of metastatic nodules were counted. FIG. 6 shows that injection with the SAGE dramatically reduced lung metastasis. Additionally, SAGE also substantially improved survival rate in mice over the month, compared to animals receiving tumor cells and only PBS alone (FIG. 7). These results, along with selectin and RAGE inhibiting activities (Table 4), indicate that SAGEs can be used as potential anti-cancer therapeutic strategies in humans, including the prevention of metastatic spread of cancer cells from tumors. In practice, SAGE therapy could be administered subcutaneously or intravenously prior to a surgical tumor resection, during the resection, and after the resection to limit attachment of any tumor cells released during surgery. SAGE therapy could also be administered subcutaneously or intravenously either prophyllactically or therapeutically to patients not undergoing oncological surgery to limit the spread of the disease.

### 5. Example 5

### SAGE Inhibition of Tumor Cell Migration

LL-37 has shown activity of stimulating the migration of various cell types and is overexpressed in ovarian, breast, and lung cancer. The above-disclosed results indicate that SAGEs can reduce metastatic melanoma progression in vivo using the highly aggressive B16F10 model for metastatic disease progression. In vitro experiments showed that SAGE can directly inhibit the growth and viability of B16F10 melanoma cells as well as three metastatic cancer cell lines, A549 (non-small cell lung cancer cells) and HCT116 (Human colon carcinoma cells), and MDA-MB-231 (Human breast cancer cells.

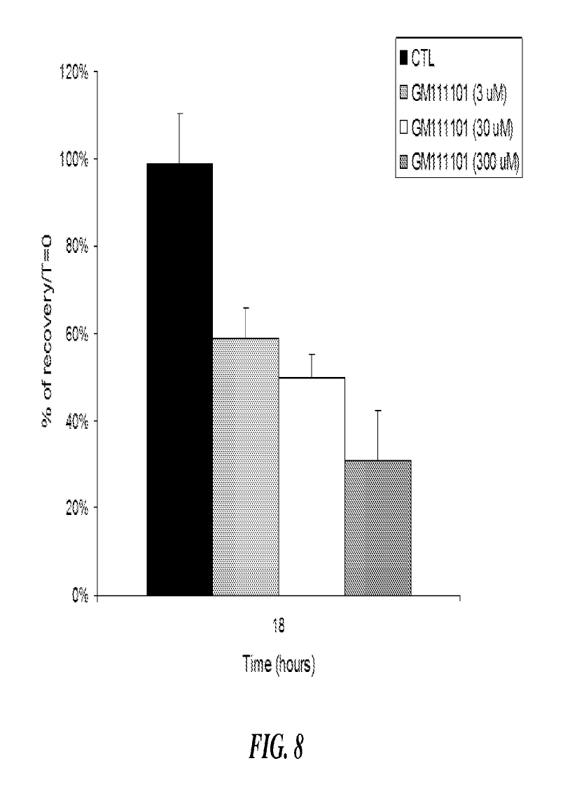

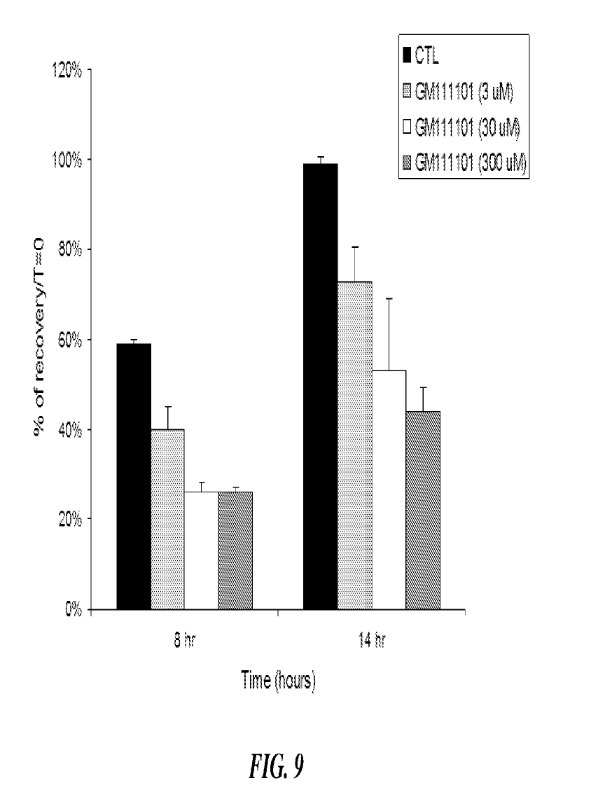

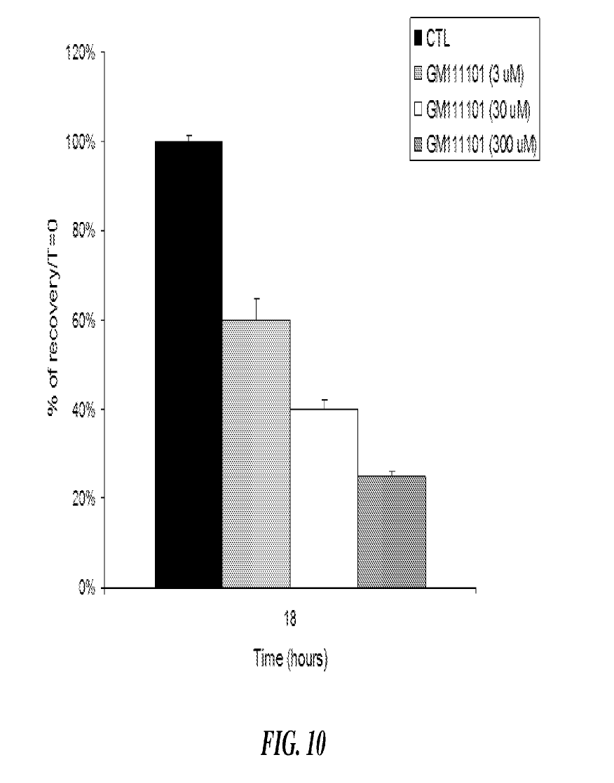

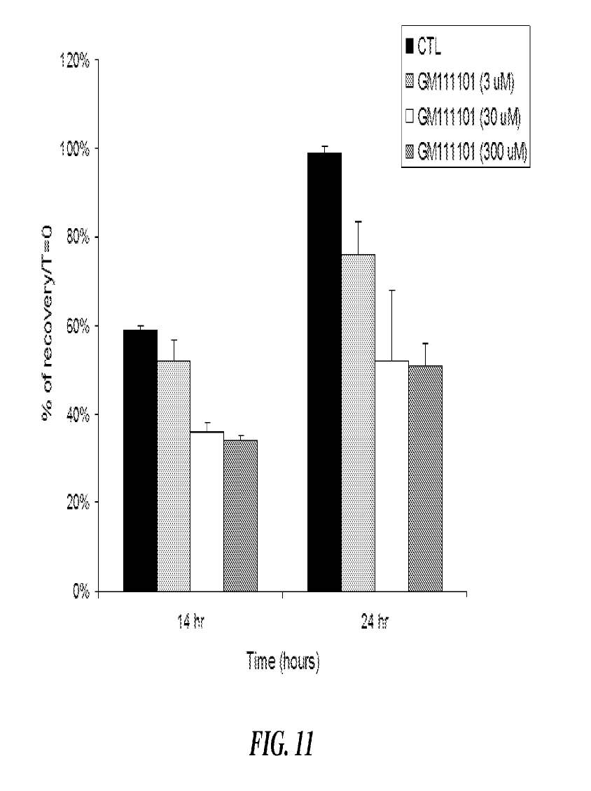

The anti-metastasis effect of a SAGE was evaluated on metastatic A549 lung cancer cells using a scratch wound assay. After treatment with different concentrations of SAGE (GM111101), cells were allowed to migrate into the denuded area for 0 and 18 hours. By 18 hours, untreated control cells completely filled the scratched area. Treatment with SAGE at 30 μM and 300 μM inhibited the A549 cell migration (FIG. 8). Migration of A549 cells was decreased by 52% (P<0.05) by 300 μM when compared with the untreated control. In the meantime, no measurable reduction in viability was observed up to 300 μM treatment, but the cancer cells were found more rounded compared to the flat untreated cells. Similar results were observed when using B16F10 melanoma cells with a more significant anti-metastatic effect (FIG. 9), and two other metastatic cancer cell lines, which were HCT116 (FIG. 10) and MDA-MB-231 (FIG. 11).

In order to investigate the capability of SAGEs to influence metastatic melanoma tumors in vivo, this efficacy was tested using the B16F10 metastatic melanoma mouse model. As previously described, the melanoma cells exhibit aggressive lung metastatic behavior when injected intravenously into B57/BL6 mice. B16F10 metastatic melanoma cells were injected into the tail vein of C57/B16 mice. Animals were subcutaneously injected on 30 minutes after intravenous 5×10⁵ cell injection with 100 μL of 10 mg/kg GM111101 and 30 mg/kg GM111101, comparing to the 30 mg/kg Heparin treatment treatment in the comparison groups and PBS in the control group. Mice were then sacrificed at or just before 28 days after the injection of B16F10 cells, and lung tissues were fixed and analyzed for the number of metastases. Micrographs (FIG. 6) and numbers of melanin-laden (black) metastasis in the lungs were calculated. Treatment with 10 mg/kg and 30 mg/kg SAGEs significantly reduced the number of pulmonary metastasis in mice as compared to the control treatment (66% reduction compared to PBS treatment group) (FIG. 5).

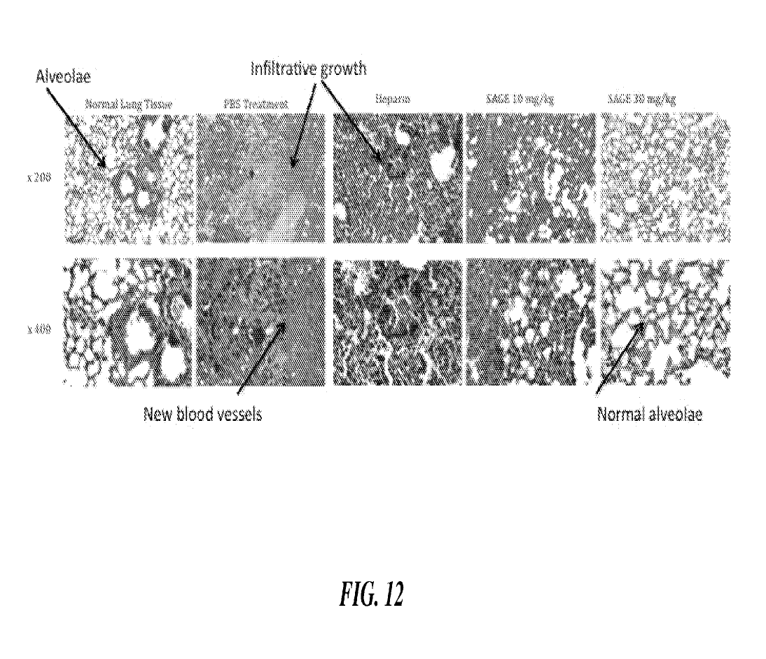

Histology demonstrates that these emergent lung metastases outgrowth significantly and induce massive angiogenesis (FIG. 12, PBS treatment), which showed infiltrative growth pattern with venous invasion. The inner portion of the tumor showed a trabecular pattern with infiltrative growth pattern. Subcutaneously administration of SAGE (10 mg/kg) revealed significant effects on the extent of lung metastatic outgrowth, with an expansive growth pattern with decreased invasion. Moreover, SAGE (30 mg/kg) treatment suppress completely lung metastatic colonization (FIG. 12), resulting in similar histology pattern as for normal lung tissue section.

In addition to the effect on pulmonary metastasis, SAGE was found to dramatically improve mice survival rate during treatment course (FIG. 7).

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

### REFERENCES

Abe R, Shimizu T, Sugawara h, Watanabe H, Nakamura H, Choei H, Sasaki N, Yamagishi S, Taeuchi M, Shimizu H. Regulation of human melanoma growth and metastasis by AGE-AGE receptor interactions. J Invest Dermatol 122:461-467, 2004. PMID 15009731

Alifano M, Benedetti G, Trisolini R. Can low-molecular-weight heparin improve outcome of patients with operable non-small cell lung cancer? An urgent call for research. Chest 126:601-607, 2004.

Altinbas M, Coskun HS, Er O, OZkan M, Eser B, Unal A, Cetin M, Soyuer S. A randomized clinical trial of combination chemotherapy with and without low-molecular-weight heparin in small cell lung cancer. J Thromb Haemost 2:1266-1271, 2004.

Bell W R, Starksen N F, Tong S, Porterfield J K. Trousseau's syndrome. Devastating coagulopathy in the absence of heparin. Am J Med 79:432-430, 1985.

Bierhaus A, Humpert P M, Morcos M, Wendt T, Chavakis T, Arnold B, Stern D M, Nawroth P P. Understanding RAGE, the receptor for advanced glycation end products. J Mol Med 83:876-886, 2005.

Bitan M, Polliack A, Zecchina G, et al. Heparanase expression in human leukemias is restricted to acute myeoloid leukemias. Exp Hematol 30:34-41, 2002.

Borsig L, Wong R, Feramisco J, Nadeau D R, Varki N M, Varki A. Heparin and cancer revisited: Mechanistic connections involving platelets, P-selectin, carcinoma mucins, and tumor metastasis. Proc Natl Acad Sci USA 98:3352-3357, 2001.

Borsig L, Wong R, Hynes R O, Varki N M, Varki A. Synergistic effects of L- and P-selectin in facilitating tumor metastasis can invovle non-mucin ligands and implicate leukocytes as enhancers of metastasis. Proc Natl Acad Sci USA 99:2193-2198, 2002.

Bralley E E, Greenspan P, Hargrove J L, Wicker L, Hartle D K. Topical anti-inflammatory activity of Polygonum cuspidatum in the TPA model of mouse ear inflammation. Journal of Inflammation 5:1, 2008. doi:10.1186/1476-9255-5-1.

Casu B, Vlodaysky I, Sanderson R D. Non-anticoagulant heparins and inhibition of cancer. Pathophysiol Haemost Thromb 36:195-203, 2007.

Chavakis T, Bierhaus A, Al-Fakhri N, Schneider D, Witte S, Linn T, Nagashima M, Morser J, Arnold B, Preissner K T and Nawroth P P. The pattern recognition receptor (RAGE) is a counterreceptor for leukocyte integrins: a novel pathway for inflammatory cell recruitment. J Exp Med 198:1507-1515, 2003. PMCID14623906

Ellerman J E, Brown C K, de Vara M, Zeh H J, Billiar T, Rubartelli A, Lotze M T. Masquerander: high mobility group box-1 and cancer. Clin Cancer Res 13:2836-2848, 2007. PMID 1704981

Friedmann Y, Vlodaysky I, Aingorn H, et al. Expression of heparanase in normal, dysplastic, and neoplastic human colonic mucosa and stroma: evidence for its role in colonic tumorigenesis. Am J Pathol 157:1167-1175, 2000.

Fryer A D, Jacoby D B. Function of pulmonary M2 muscarinic receptors in antigen-challenged guinea pigs is restored by heparin and poly-L-glutamate. J Clin Invest 90:2292-2298, 1992. PMCID1281829

Gao F, Koenitzer J R, Tobolewski J M, Jiang D, Liang J, Noble P W and Oury T D. Extracellular superoxide dismutase inhibits inflammation by preventing oxidative fragmentation of hyaluronan. J Biol Chem. 283:6058-6066, 2008. PMCID18165226

Gao Y, Wei M, Meng S, Ba X, Hao S, Zeng X. Chemically modified heparin inhibits in vitro adhesion of nonsmall cell lung cancer cells to P-selectin. J Cancer Res Clin Oncol 132:257-264, 2006.

Ginanth S, Menczer J, Friedmann Y, et al. Expression of heparanase, Mdm2, and erbB2 in ovarian cancer. Int J Oncol 18:1133-1144, 2001.

Gohji K, Okamoto M, Kitazawa S, et al. Haparanse protein and gene expression in bladder cancer. J Urol 166:1286-1290, 2001.

Guerrini M, Beccati D, Shriver Z, Naggi A, Viswanathan K, Bisio A, Capila I, Lansing J C, Guglieri S, Fraser B, Al-Hakim A, Gunay N S, Zhang Z, Robinson L, Buhse L, Nasr M, Woodcock J, Langer R, Venkataraman G, Linhardt R J, Casu B, Toni G and Sasisekharan R. Oversulfated chondroitin sulfate is a contaminant in heparin associated with adverse clinical events. Nat Biotech 26:669-675, 2008. PMC Journal—In Process

Harpio R, Einarsson R. S100 proteins as cancer biomarkers with focus on S100B in malignant melanoma. Clin Biochem 37:512-518, 2004. PMID 15234232

Hirsh J, Raschke R. Heparin and low-molecular weight heparin: The seventh ACCP conference on antithrombotic and thrombolytic therapy. Chest 126:188-203, 2004.

Howes K A, Liu Y, Dunaief J L, Milam A, Frederick J M, Marks A and Baehr W. Receptor for advanced glycation end products and age-related macular degeneration. Invest Opthalmol Vis Sci 45:3713-3720, 2004. PMCID15452081

Ilan N, Elkin M, Vlodaysky I. Regulation, function and clinical significant of heparanse in cancer metastasis and angiogenesis. Int J Biochem Cell Biol 38:2018-2139, 2006. PMID 16901744

Jiang D, Liang J and Noble P W. Hyaluronan in tissue injury and repair. Annu Rev Cell Dev Biol 23:435-461, 2007. PMCID17506690

Juni P, Reichenbach S, Trelle S, Tschannen B, Wandel S, Jordi B, Zullig M, Guetg R, Hauselmann H J, Schwarz H, Theiler R, Ziswiler H R, Dieppe P A, Villiger P M and Egger M for the Swiss Viscosupplementation Trial Group. Efficacy and safety of intra-articular hylan or hyaluronic acids for osteoarthritis of the knee: a randomized controlled trial. Arthritis Rheum 56:3610-3619, 2007. PMCID17968921

Kakkar A K, Levine N M, Kadziola Z, Lemoine N R, Low V, Patel H K, rustin G, Thomas M, Quigley M, Williamson R C N. Low molecular weight heparin, therapy with dalteparin, and survivial in advanced cancer: the Fragmin Advanced Malignancy Outcome Study (FAMOUS). J Clin Oncol 22:1944-1948, 2004.

Kennedy T P. Method and medicament for sulfated polysaccharide treatment of heparin-induced thrombocytopenia (HIT) syndrome. U.S. Pat. No. 7,468,358. Issued Dec. 23, 2008.

Kishimoto T K, Viswanathan K, Ganguly T, Elankumaran S, Smith S, Pelzer K, Lansing J C, Sriranganathan N, Zhao G, Galcheva-Gargova Z, Al-Hakim A, Bailey G S, Fraser B, Roy S, Rogers-Cotrone T, Buhse L, Whary M, Fox J, Nasr M, Dal Pan G J, Shriver Z, Langer R S, Venkataranam G, Austen K F, Woodcock J and Sasisekharan R. Contaminated heparin associated with adverse clinical events and activation of the contact system. N Engl J Med 358:2457-2467, 2008. PMCID18434646

Klerk C P W, Smorenburg S M, Otten H-M, Lensing A W A, Prins M H, Piovells F, Prandoni P, Bos M M E M, Richel D J, van Tienhoven G, Buller H R. The effect of low molecular weight heparin on survival in patients with advanced malignancy. J Clin Oncol 23:2130-2135, 2005.

Koenig A, Norgard-Sumnicht K, Linhardt R, Varki A. Differential interactions of heparin and heparan sulfate glycosaminoglycans with the selectins Implications for the use of unfractionated and low molecular weight heparins as therapeutic agents. J Clin Invest 101:877-889, 1998.

Koliopanos A, Friess H, Kleeff J, et al. Heparanase expression in primary and metastatic pancreatic cancer. Cancer Res 61:4655-4659, 2001.

Kragh M, Binderup L, Big Hjarnaa P-J, Bramm E, Johansen K B, Petersen C F. Non-anti-coagulant heparin inhibits metastasis but not primary tumor growth. Oncol Reports 14:99-104, 2005.

Kragh M, Loechel F. Non-anti-coagulant heparins: A promising approach for prevention of tumor metastasis. Int J Oncol 27:1159-1167, 2005.

Lapierre F, Holme K, Lam L, Tressler R J, Storm N, Wee J, Stack R J, Castellot J, Tyrrell D J. Chemical modifications of heparin that diminish its anticoagulant but preserve its heparanase-inhibitory, angiostatic, anti-tumor and anti-metastatic properties. Glycobiol 6:355-366, 1996.

Lebeau B, Chatang C, Brechot J-M, Capron F, Dautzenberg B, Dalisements C, Mornet M, Brun J, Hurdebourcq J-P, Lemaire E, for the “Petites Cellules” Group. Subcutaneous heparin treatment increases survival in small cell lung cancer. Cancer 74:38-45, 1994.

Lee A Y Y, Rickles F R, Julian J A, Gent M, Baker R I, Bowden C, Kakkar A K, Prins M, Levine M N. Randomized comparison of low molecular weight heparin and coumarin derivatives on the survival of patients with cancer and venous thromboembolism. J Clin Oncol 23:2123-2129, 2005.

Levine M. Managing thromboembolic disease in the cancer patient: efficacy and safety of antithrombotic treatment options in patients with cancer. Cancer Treat Rev 28:145-149, 2002.

Lider O, Baharav E, Mekori Y A, Miller T, Naparstek Y, Vlodaysky I, Cohen I R. Suppression of experimental autoimmune diseases and prolongation of allograft survival by treatment of animals with low doses of heparins. J Clin Invest 83:752-756, 1989.

Lin L, Park S, Lakatta E G. RAGE signaling in inflammation and arterial aging. Front Biosci 14:1403-1413, 2009.

Logsdon C D, Tuentes M K, Huang E H, Arumugam T. RAGE and RAGE ligands in cancer. Curr Mol Med 7:777-789, 2007. PMID 18331236

Loynes J T, Zacharski L R, Rigas. Regression of metastatic non-small cell lung cancer with low molecular weight heparin. Thromb Haemost 88:686, 2002.

Ma Y-Q, Geng J-G. Heparan sulfate-like proteoglycans mediate adhesion of human malignant melanoma A375 cells to P-selectin under flow. J Immunol 165-558-565, 2000.

Mahtouk K, Hose D, Raynaud P, Hundemer M, Jourdan M, Jourdan E, Pantesco V, Baudard M, De Vos J, Larroque M, Moehler T, Rossi J-F, Reme T, Goldschmidt H, Klein B. Heparanase influences expression and shedding of syndecan-1, and its expression by the bone marrow environment is a bad prognostic factor in multiple myeloma. Blood 109:49114-4923, 2007.

Mathis G. Probing molecular interactions with homogeneous techniques based on rare earth cryptates and fluorescence energy transfer. Clin Chem 41:1391-1397, 1995.

Maxhimer J B, Quiros R M, Stewart R, et al. Heparanase-1 expression is associated with the metastatic potential of breast cancer. Surgery 132:326-333, 2002.

McMorran B J, Patat S A, Carlin J B, Grimwood K, Jones A, Armstrong D S, Galati J C, Cooper P J, Byrnes C A, Francis P W, Robertson C F, Hume D A, Borchers C H, Wainwright C E and Wainwright B J. Novel neutrophil-derived proteins in bronchoalveolar lavage fluid indicate an exaggerated inflammatory response in pediatric cystic fibrosis patients. Clin Chem 53:1782-1791, 2007. PMCID17702859

Mielicki W P, Tenderena M, Rutkowski P, Chounowski K. Activation of blood coagulation and the activity of cancer procoagulant (EC 3.4.22.26) in breast cancer patients. Cancer Lett 146:61-66, 1999.

Myint K-M, Yamamoto Y, Doi T, Kato I, Harashima A, Yonekura H, Watanabe T, Shinohara H, Takeuchi M, Tsuneyama K, Hasimoto N, Asano M, Takasawa S, Okamoto H and Yamamoto H. RAGE control of diabetic nephropathy in a mouse model. Effects of RAGE gene disruption and administration of low-molecular weight heparin. Diabetes 55:2510-2522, 2006. PMCID16936199

Naggi A, Casu B, Perez M, et al. Modulation of the heparanase-inhibiting activity of heparin through selective desulfation, graded N-acetylation, and glycol splitting. J Biol Chem 280:121-3-12113, 2005.

Ono K, Ishihara M, Ishikawa K, Ozeiki Y, Deguchi H, Sato M, Hashimoto H, Saito Y, Yura H, Kurita A, Maehara T. Periodate-treated, non-anticoagulant heparin-carrying polystyrene (NAC-HCPS) affects angiogenesis and inhibits subcutaneous induced tumour growth and metastasis to the lung. Brit J Cancer 86:1803-1812, 2002.

Prechel M M, McDonald M K, Jeske W P, Messmore H L, Walenga J M. Activation of platelets by heparin-induced thrombocytopenia antibodies in the serotonin release assay is not dependent on the presence of heparin. J Thromb Haemost 3:2168-2175, 2005.

Purushothaman A, Chen L, Yang Y, Sanderson R D. Heparanase stimulation of protease expression implicates it as a master regulator of the aggressive tumor phenotype in myeloma. J Biol Chem 283:32628-32636, 2008.

Ramasamy R, Vannucci, S J, Du Yan S S, Herold K, Yan S F, Schmidt A M. Advanced glycation end products and RAGE: a common thread in aging, diabetes, neurodegeneration, and inflammation. Glycobiol 15:16R-28R, 2005.

Sack G H Jr, Levin J, Bell W R. Trousseau's syndrome and other manifestations of chronic disseminated coagulopathy in patients with neoplasms: clinical, pathophysiologic, and therapeutic features. Medicine (Baltimore) 56:1-37, 1977.

Samuels M A, King M E, Balis U. Case records of the Massachusetts General Hospital. Weekly clinicopathological exercise. Case 31-2002. A 61-year-old man with headache and multiple infarcts. N Engl J Med 347:1187-1194, 2002.

Schmidt A M, Yan S D, Yan S F and Stern D M. The multiligand receptor RAGE as a progression factor amplifying immune and inflammatory responses. J Clin Invest 108: 949-955, 2001. PMCID11581294

Smorenburg S M, van Noorden C J F. The complex effects of heparins on cancer progression and metastasis in experimental studies. Pharmacol Rev 53:93-105, 2001.

Stevenson J L, Choi S H, Varki A. Differential metastasis inhibition by clinically relevant levels of heparins—correlation with selectin inhibition, not antithrombotic activity. Clin Cancer Res 11:7003-7011, 2005.

Stevenson J L, Varki A, Borsig 1. Heparin attenuates metastasis mainly due to inhibition of P- and L-selectin, but non-anticoagulant heparins can have additional effects. Thromb Res 120:S107-5111, 2007.

Toyoshima M, Nakajima M. Human hapranase: purification, characterization, cloning, and expression. J Biol Chem 274:24153-24160, 1999.

Trousseau A. Plegmasia alba dolens. Lectures on clinical medicine, delivered at the hotel-Dieu, Paris. P V Bazire, editor and translator. The New Sydenham Society Publications, London, United Kingdom 55:281-332, 1868.

Varki A. Trousseau's syndrome: multiple definitions and multiple mechanisms. Blood 110:1723-1729, 2007.

Wahrenbrock M, Borsig L, Le D, Varki N, Varki A. Selectin-mucin interactions as a probable molecular explanation for the association of Trousseau syndrome with mucinous adenocarcinomas. J Clin Invest 112:853-862, 2003.

Walenga J M, Jeske W P, Prechel M M, Makhos M. Newer insights on the mechanism of heparin-induced thrombocytopenia. Semin Thromb Hemost 30(Suppl 1): 57-67, 2004.

Wang L, Brown J R, Varki A and Esko J D. Heparin's anti-inflammatory effects require glucosamine 6-O-sulfation and are mediated by blockade of L- and P-selectins. J Clin Invest 110:127-136, 2002. PMCID12093896

Wei M, Tai G, Gao Y, Li N, Huang B, Zhou Y, Hao S, Zeng X. Modified heparin inhibits P-selectin-mediated cell adhesion of human colon carcinoma cells to immobilized platelets under dynamic flow conditions. J Biol Chem 279:29202-29210, 2004.

Yamasaki K, Di Nardo A, Bardan A, Murakami M, Ohtake T, Coda A, Dorschner R A, Bonnart C, Descargues P, Hovnanian A, Morhenn V B, Gallop R L. Increased serine protease activity and cathelicidin promotes skin inflammation in rosacea. Nat Med 13:975-980, 2007. PMCID17676051

Yan S S, Wu Z-Y, Zhang H P, Furtado G, Chen X, Yan S F, Schmidt A M, Brown C, Stern A, LaFaille J, Chess L, Stem D M and Jiang H. Suppression of experimental autoimmune encephalomyelitis by selective blockade of encephalitogenic T-cell infiltration of the central nervous system. Nat Med 9:287-293, 2003. PMCID12598893

Yan Y, MacLeod V, Dai Y, Khotskaya-Sample Y, Shriver Z, Venkataraman G, Sasisekharan R, Naggi A, Toni G, Casu B, Vlodaysky I, Suva L J, Epstein J, Yaccoby S, Shaughnessy J D Jr, Barlogie B, Sanderson R D. The syndecan-1 heparan sulfate proteoglycan is a viable target for myeloma therapy. Blood 110:2041-2048, 2007.

Yang Y, MacLeod V, Bendre M, Huang Y, Theus A M, Miao H-Q, Kussie P, Yaccoby S, Epstein J, Suva L J, Kelly T, Sanderson R D. Heparanase promotes the spontaneous metastasis of myeoloma cells to bone. Blood 105′1303-1309, 2005.

Yang Y, MacLeod V, Miao H-Q, Zhan F, Shaughnessy J D Jr, Sawyer J, Li J-P, Zcharia E, Vlodaysky I, Sanderson R D. Heparanase enhances syndecan-1 shedding. A novel mechanism for sstimulation of tumor growth and metastasis. J Biol Chem 282:13326-13333, 2007.

Yip G Q W, Smollich M, Gotte M. Therapeutic value of glycosaminoglycans in cancer. Mol Cancer Ther 5:2139-2148, 2006.

Yoshitomi Y, Nakanishi H, Kusano Y, Munesue S, Oguri K, Tatematsu M, Yamashina I, Okayama M Inhibition of experimental lung metastases of Lewis lung carcinoma cells by chemically modified heparin with reduced anticoagulant activity. Cancer Lett 207:165-174, 2004.

Zacharski L R, Schned A R, Sorenson G D. Occurrence of fibrin and tissue factor antigen in human small cell carcinoma of the lung. Cancer Res 43:3963-3968, 1983.

Zhang W, Liu J N, Tan X Y. Vaccination with xenogeneic tumor endothelial proteins isolated in situ inhibits tumor angiogenesis and spontaneous metastasis. Int J Cancer 125:124-132, 2009.

What is claimed is:

1. A method of treating cancer, comprising administering to a subject in need thereof a therapeutically effective amount of a composition comprising a modified hyaluronan or the pharmaceutically acceptable salt or ester thereof, wherein at least one primary C-6 hydroxyl proton of the N-acetyl-glucosamine residue is substituted with an alkyl group or fluoroalkyl group, and wherein at least one hydroxyl proton of hyaluronan is substituted with a sulfate group.

2. The method of claim 1, wherein the alkyl group comprises a C₁-C₁₀ branched or straight chain alkyl group.

3. The method of claim 1, wherein the alkyl group comprises methyl, ethyl, propyl, iso-propyl, butyl, pentyl, or hexyl.

4. The method of claim 1, wherein the alkyl group is methyl.

5. The method of claim 1, wherein the fluoroalkyl group comprises at least one trifluoromethyl group.

6. The method of claim 1, wherein the fluoroalkyl group comprises the formula —CH₂(CF₂)ₙCF₃, wherein n is an integer from 0 to 10.

7. The method of claim 6, wherein n is 1, 2, 3, 4, or 5.

8. The method of claim 1, wherein the pharmaceutically acceptable ester is a prodrug.

9. The method of claim 1, wherein at least 1% of the primary C-6 hydroxyl protons of the N-acetyl-glucosamine residue are substituted with an alkyl group or fluoroalkyl group.

10. The method of claim 1, wherein from 1% to 100% of the primary C-6 hydroxyl protons of the N-acetyl-glucosamine residue are substituted with an alkyl group or fluoroalkyl group.

11. The method of claim 1, wherein at least one C-2 hydroxyl proton or C-3 hydroxyl proton is substituted with an alkyl group or fluoroalkyl group.

12. The method of claim 1, wherein the hyaluronan has a molecular weight greater than 10 kDa prior to alkylation or fluoroalkylation.

13. The method of claim 1, wherein the hyaluronan has a molecular weight from 40 kDa to 2,000 kDa prior to alkylation or fluoroalkylation.

14. The method of claim 1, wherein at least one C-2 hydroxyl proton, C-3 hydroxyl proton, C-4 hydroxyl proton, C-6 hydroxyl proton, or any combination thereof is substituted with a sulfate group.

15. The method of claim 1, wherein the C-4 and/or C-6 hydroxyl protons of the N-acetyl-glucosamine residue of hyaluronan are substituted with a sulfate group

16. The method of claim 1, wherein the compound has a degree of sulfation from 0.5 to 3.5 per disaccharide unit.

17. The method of claim 1, wherein the alkyl group is methyl and (1) at least one C-2 hydroxyl proton and/or C-3 hydroxyl proton present on a glucuronic ring of hyaluronan is substituted with a sulfate group, (2) at least one C-4 and/or C-6 hydroxyl protons of the N-acetyl-glucosamine residue is substituted with a sulfate group, or any combination thereof.

18. The method of claim 17, wherein the compound has a molecular weight of 2 kDa to 10 kDa.

19. The method of claim 1, wherein the fluoroalkyl group is —CH₂CF₂CF₃ or —CH₂CF₂CF₂CF₃ and at least one C-2 hydroxyl proton and/or C-3 hydroxyl proton present on a glucuronic ring of hyaluronan is substituted with a sulfate group.

20. The method of claim 1, wherein the treatment inhibits the spread of tumor metastasis.

21. The method of claim 1, wherein the treatment reduces the size of the primary tumor or reduces the ability of the cells from the primary tumor to form new tumors.

22. The method of claim 1, wherein the subject has been diagnosed with a metastatic tumor.

23. The method of claim 22, wherein the tumor is prostate cancer, melanoma, pancreatic cancer, kidney cancer, liver cancer, breast cancer, lung cancer, colon cancer, ovarian cancer, a gastrointestinal cancer, or a myeloma.

24. The method of claim 1, wherein the modified hyaluronan inhibits P-selectin and L-selectin and Receptor for Advanced Glycation End-products (RAGE) but has low anti-coagulant activity compared to heparin.
